(19) United States
(12) Patent Application Publication
(10) Pub. No.: US 2023/0056196 A1
(43) Pub. Date: Feb. 23, 2023

(54) DENTAL DISEASE TREATING DEVICE AND METHOD FOR TREATING DENTAL DISEASE

(71) Applicant: FEAGLE CO., LTD, Yangsan-si (KR)

(72) Inventors: Hyunyoung LEE, Yangsan-si (KR); Jeonghae CHOI, Busan (KR); Byulbora CHOI, Yangsan-si (KR)

(73) Assignee: FEAGLE CO., LTD, Yangsan-si (KR)

(21) Appl. No.: 17/889,356

(22) Filed: Aug. 16, 2022

(30) Foreign Application Priority Data
Aug. 17, 2021 (KR) 10-2021-0107939

(51) Int. Cl. A61C 19/06; H05H 1/24

(52) U.S. Cl. CPC A61C 19/06; H05H 1/2431; H05H 1/2443; H05H 2245/36

(57) ABSTRACT

Disclosed are a dental disease treating device, and more particularly, to a dental disease treating device that may generate high-density plasma that is required for a treatment of a dental disease, such as periodontitis, and minimize generation of ozone as well, and a method for treating a dental disease. The dental disease treating device that generates plasma for treating a dental disease, includes a handpiece body having a space part in an interior thereof, a plasma generating part mounted in the space part of the handpiece body, and that generates the plasma by exciting a gas in a plasma generating space in an interior thereof, and a tip part detachably coupled to an end nozzle of the handpiece body or the plasma generating part, and the tip part includes a passage for irradiating the plasma to a local portion while inhibiting oxygen from being introduced into the plasma generating space.

### CROSS-REFERENCE TO RELATED APPLICATIONS

This application claims priority under 35 U.S.C. § 119 to Korean Pat. Application No. 10-2021-0107939 filed on Aug. 17, 2021, in the Korean Intellectual Property Office, the disclosures of which are incorporated by reference herein in their entireties.

### BACKGROUND

Embodiments of the present disclosure described herein relate to a dental disease treating device, and more particularly, to a dental disease treating device that may generate high-density plasma that is required for a treatment of a dental disease, such as periodontitis, and minimize generation of ozone as well, and a method for treating a dental disease.

The present disclosure was derived from a study performed as a part of a foundation & growth technology development (R&D) (unique project No. 1425148330, project No. S2848389, study project name: Development of Paronichia and Periodontitis of Implant using Cold plasma, project management institute: Small and Medium Business Information Agency, project performance institute: Peegle, study period: Jul. 15, 2020 to Jul. 14, 2022) of the Small and Medium Business Administration. Meanwhile, the present disclosure has no property benefit of Korean Government in all aspects.

A conventional atmospheric plasma gas generating device includes a plate (electrode plate) and a central electrode connected to a power supply line for generating plasma and an insulator for preventing a short-circuit between the plate and the central electrode, and includes a gas supply pipe for discharging the plasma gas. Plasma gas generating devices disclosed in Korean Pat. No. 10-0828590, Korean Pat. No. 10-0807806, and Korean Pat. No. 10-0723019, and the like are advantageous for welding or surface-treatment of machined products because plasma is flame-discharged in jet forms, but are not suitable for surface-treatments of medical instruments or medical devices for treating human bodies.

Sterilization performances of cold plasma for various microorganisms in oral cavities have been reported, but oxygen is easily introduced into a plasma generating device and ozone is generated together with plasma, and the ozone may be introduced into a respiratory system of a human body to cause respiratory diseases. Accordingly, the conventional plasma generating device is not suitable for purposes such as treatment of a periodontitis, and to directly apply a sterilization performance of cold plasma to an oral cavity that is a respiratory system, a technology of inhibiting generation of ozone when the plasma is generated while effectively delivering the plasma for treating the periodontitis to the oral cavity of the human body. The background technology described above does not mean a technology that was known the filing date of the present disclosure, and should be construed to describe the background technology of the present disclosure.

### SUMMARY

An aspect of the present disclosure provides a dental disease treating device that may generate high-density plasma that is required for a treatment of a dental disease, such as periodontitis, and minimize generation of ozone as well, and a method for treating a dental disease.

Another aspect of the present disclosure provides a dental disease treating device that may effectively treat various dental diseases while securing a safety of oral cells, and a method for treating a dental disease.

The technical problems that are to be solved by the present disclosure are not limited to the above-mentioned ones, and the other technical problems that have not been mentioned will be clearly understood from the following description by an ordinary person in the art, to which the present disclosure pertains.

According to an aspect of the present disclosure, a dental disease treating device that generates plasma for treating a dental disease, the dental disease treating device includes a handpiece body having a space part in an interior thereof, a plasma generating part mounted in the space part of the handpiece body, and that generates the plasma by exciting a gas in a plasma generating space in an interior thereof, and a tip part detachably coupled to an end nozzle of the handpiece body or the plasma generating part, and the tip part includes a passage for irradiating the plasma to a local portion while inhibiting oxygen from being introduced into the plasma generating space.

The dental disease treating device may further include a grip part mounted to surround an outer peripheral surface of the handpiece body, and coupled to the handpiece body to be separable.

A tip coupling part may be formed at an end of the handpiece body, the tip coupling part may be provided at an inner diameter portion of a tip end of the grip part, and the end nozzle may be formed in an interior of the tip coupling part. The tip part may have a Luer connector structure.

The tip part may include a tip body having a cylindrical shape, a plasma delivery pipe communicated with one side of the tip body, and having a cylindrical shape that is thinner than the tip body, and a coupling hole formed on an opposite side of the tip body to be coupled to the end nozzle.

The tip part may inhibit generation of ozone when the plasma is generated, by preventing ambient air from penetrating into the plasma generating space.

The plasma generating part may include a cylindrical body having an interior space, an inner electrode inserted into the interior space of the body, and including a cylindrical electrode body, and an outer electrode formed on an outer peripheral surface of the body through metalizing.

A gas introduction pipe including a first passage, through which the gas for generating the plasma is introduced, may be connected to a rear end of the electrode body, and a tip end of the electrode body has a conical shape.

The first passage may be communicated with an interior passage of the electrode body, and a plurality of gas discharge holes for discharging the gas introduced through the first passage toward the plasma generating space may be radially formed in the electrode body.

A first coupling part having a first opening may be formed at one end of the body, a second coupling part having a second opening may be formed at an opposite end of the body, a coupling recess that is to be coupled to the body may be formed in the second coupling part, and a coupling boss formed on an inner surface of the handpiece body may be coupled to the coupling recess.

The inner electrode may be grounded, and an AC voltage for exciting the gas is applied to the outer electrode. The plasma generating part may further include an elastic pad interposed between the second coupling part of the body, and a flange part of the inner electrode, and the elastic pad may seal an aperture between the body and the inner electrode such that the gas is neither discharged nor introduced through the aperture between the body and the inner electrode.

The body, the outer electrode, the inner electrode, and the tip part may be designed to satisfy parameters of Equations 1 to 6 as follows such that a volume of the generated plasma and an acceleration distance of electrons are increased, high-density plasma is effectively delivered to an outer area, and generation of ozone is inhibited,

$$0.5 \leq \text{L1/L2} \leq \text{1}$$

$$0.5 \leq \text{D2/D1} \leq \text{0} \text{.8}$$

$$0.5 \leq \text{L3/L2} \leq \text{1}$$

$$0.5 \leq \text{L3/L2} \leq \text{1}$$

$$16 \text{gauge} \leq \text{D3} \leq \text{20} \text{gauge}$$

$$2 \text{cm < L4 < 3} \text{.5 cm, and}$$

in Equations 1 to 6, L1 is a length of the outer electrode, L2 is a length of the inner electrode, L3 is a distance between the front end of the inner electrode and the front surface of the body, L4 is a length of the tip part, D1 is a diameter of an inner passage of the body, D2 is a diameter of the inner electrode, and D3 is a minimum diameter of a passage of the tip part.

The length of the inner electrode may be 10 mm to 20 mm, the length of the outer electrode may be 5 mm to 20 mm, the diameter of the inner electrode may be 2 mm to 4 mm, the diameter of the inner passage of the body may be 3 mm to 5 mm, and a distance between a front end of the inner electrode and a front surface of the body may be 1 mm to 4 mm.

According to another aspect of the present disclosure, a method for treating a dental disease, by which a dental disease of a target object is treated by using the dental disease treating device is provided.

### BRIEF DESCRIPTION OF THE FIGURES

The above and other objects and features of the present disclosure will become apparent by describing in detail embodiments thereof with reference to the accompanying drawings.

FIG. 1 is a perspective view of a dental disease treating device according to an embodiment of the present disclosure;

FIG. 2 is a perspective view of a handpiece that constitutes a dental disease treating device according to an embodiment of the present disclosure;

FIG. 3 is a view illustrating an attachment/detachment structure of a tip part that constitutes a dental disease treating device according to an embodiment of the present disclosure;

FIG. 4 is a perspective view of a plasma generating part that constitutes a dental disease treating device according to an embodiment of the present disclosure;

FIG. 5 is an exploded perspective view of a plasma generating part that constitutes a dental disease treating device according to an embodiment of the present disclosure;

FIG. 6 is a cross-sectional view of a plasma generating part that constitutes a dental disease treating device according to an embodiment of the present disclosure;

FIG. 7 is a picture depicting a dental disease treating device according to an embodiment of the present disclosure;

FIG. 8 is a graph illustrating plasma active species elements generated by a dental plasma generating device, on which no tip is mounted;

FIG. 9 is a picture depicting a result obtained by performing surface sterilizing treatments on four species of microorganisms by using a plasma device, on which no tip is mounted;

FIG. 10 is a picture depicting a result obtained by performing surface sterilizing treatments on microorganisms by using a cold plasma device, on which no tip is mounted, and a cold plasma device, on which a tip is mounted;

FIG. 11 is a picture depicting a result obtained by cultivating microorganisms for five hours after a cold plasma treatment of the microorganisms;

FIG. 12 is a graph depicting values obtained by analyzing bacterium growth inhibiting performances when microorganisms are cultivated for five hours after a cold plasma treatment of the microorganisms;

FIG. 13 is a picture depicting a result obtained by cultivating microorganisms for twenty four hours after a cold plasma treatment of the microorganisms;

FIG. 14 is a picture depicting a result obtained by cultivating microorganisms for twenty four hours after a cold plasma treatment of the microorganisms;

FIG. 15 is a graph depicting a result obtained by comparing growth inhibiting performances by cold plasma treatments of four species of microorganisms according to whether a tip is mounted;

FIG. 16 is a graph depicting changes in a concentration of H₂O₂ of a culture medium when a cold plasma treatment is performed on a microorganism culture medium;

FIG. 17 is a graph depicting changes in a concentration of H₂O₂ of a culture medium during a cold plasma treatment according to whether a tip is mounted;

FIG. 18 is a graph depicting changes in a concentration of a nitrite of a liquid culture medium during a cold plasma treatment;

FIG. 19 is a view illustrating changes in inhibition of growth of microorganisms according to a cold plasma treatment and application of N-acetylcysteine;

FIG. 20 is a picture depicting a change in a surface sterilizing treatment for microorganisms according to a length of a capillary tip and a cold plasma treatment time period;

FIG. 21 is an exemplary view of various capillary tips used in experiments;

FIG. 22 is a picture depicting a change in a surface sterilizing treatment for microorganisms according to a length of a micro-capillary tip and a cold plasma treatment time period;

FIG. 23 is an exemplary view of various micro-capillary tips used in experiments;

FIG. 24 is a picture depicting an experiment through observation of surface sterilizing performances of cold plasma by installing a mesh between a cold plasma treating device and a microorganism solid-culture medium;

FIG. 25 is a picture depicting an experimental result through observation of surface sterilizing performances of cold plasma by installing a mesh between a cold plasma treating device and a microorganism solid-culture medium;

FIG. 26 is a graph depicting changes in generated H₂O₂ according to types of meshes during a cold plasma treatment;

FIG. 27 is a picture depicting a result through observation of an effective range of sterilizing performances when a cold plasma treatment was made at a fixed location and at variable locations;

FIG. 28 is a picture depicting changes in a shape of a BRIC according to changes of a length and an inner diameter of the tip during a cold plasma treatment;

FIG. 29 is a graph depicting changes in a concentration of H₂O₂ according to a length and an inner diameter of a tip during a cold plasma treatment;

FIG. 30 is a picture of oral periodontal cells, on which a non-ozone cold plasma treatment was performed by a dental disease treating plasma device according to an embodiment of the present disclosure;

FIG. 31 is a view illustrating a cell viability measurement result of oral periodontal cells, on which a non-ozone cold plasma treatment was performed by a dental disease treating plasma device according to an embodiment of the present disclosure;

FIG. 32 is a view illustrating changes in an inflammation inducing factor during a non-ozone cold plasma treatment by a dental disease treating plasma device according to an embodiment of the present disclosure;

FIG. 33 is a view illustrating changes in a 1L-1β mRNA gene expression inhibiting effect during a non-ozone cold plasma treatment by a dental disease treating plasma device according to an embodiment of the present disclosure;

FIG. 34 is a view illustrating changes in a 1L-6 mRNA gene expression inhibiting effect during a non-ozone cold plasma treatment by a dental disease treating plasma device according to an embodiment of the present disclosure;

FIG. 35 is a view illustrating changes in a TNF-amRNA gene expression inhibiting effect during a non-ozone cold plasma treatment by a dental disease treating plasma device according to an embodiment of the present disclosure;

FIG. 36 is a view illustrating changes in proteins during a plasma treatment by a dental disease treating plasma device according to an embodiment of the present disclosure;

FIG. 37 is a view illustrating an experimental process for verifying a periodontitis treatment performance by a dental disease treating device according to an embodiment of the present disclosure;

FIG. 38 is a micro CT analysis result using oral tissues extracted after an animal experiment is finished;

FIG. 39 is a view illustrating a hematoxylin-eosin dyeing result using oral tissues after an animal experiment is finished;

FIGS. 40 and 41 are views illustrating an RT-PCT study result using RNAs extracted from an experimental tissue;

FIG. 42 is a view illustrating an IHC result performed on CD68 proteins by using a tissue of an animal experiment; and

FIG. 43 is a view illustrating a TRAP assay performance result using a tissue of an animal experiment.

### DETAILED DESCRIPTION

Hereinafter, embodiments of the present disclosure will be described in more detail with reference to the accompanying drawings. The embodiments of the present disclosure may be modified in various forms, and the scope of the present disclosure should not be construed to be limited to the following embodiments. The embodiments of the present disclosure are provided to describe the present disclosure for an ordinary person in the art more completely.

It is explained in advance that the same reference numerals are given to the same elements even though the elements are in other drawings when the reference numerals are given to the elements of the drawings and the elements in the other drawings may be cited if necessary when the corresponding drawing is described. Parts (a gas supply pipeline, an electric circuit, or a controller) that are not relevant to a description may be omitted in a detailed description of the present disclosure to clearly describe the present disclosure.

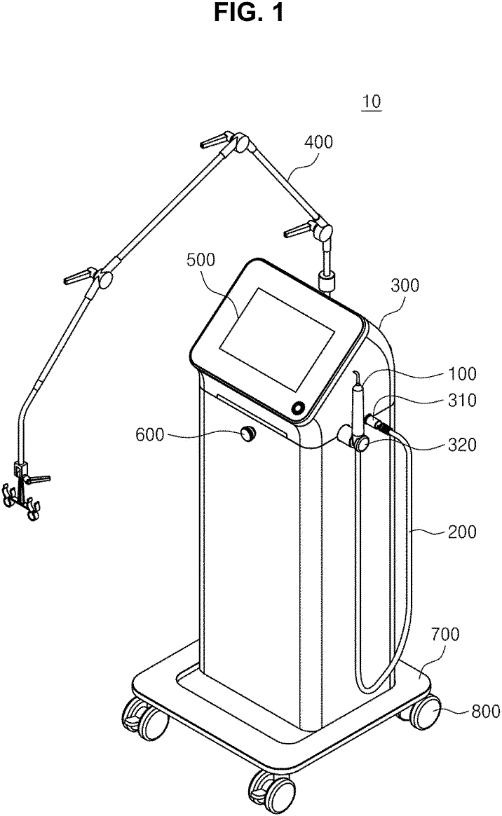

FIG. 1 is a perspective view of a dental disease treating device according to an embodiment of the present disclosure. Referring to FIG. 1, a dental disease treating device 10 according to an embodiment of the present disclosure may include a handpiece 100 corresponding to a plasma generating device that generates cold plasma for treating a dental disease such as periodontitis.

The handpiece 100 may receive a gas (for example, argon) for generating plasma through a supply line 200. The supply line 200 may be coupled to a coupling part 310 provided on a side surface of a body 300 to receive a gas from a gas supply part provided in the body 300 and deliver the gas to the handpiece 100.

The body 300 may be provided with holding arms 320 and 400 for holding the handpiece 100, a monitor 500 that allows a touch input, and an emergency stop switch 600. The body 300 may be supported on a support 700 including wheels 800.

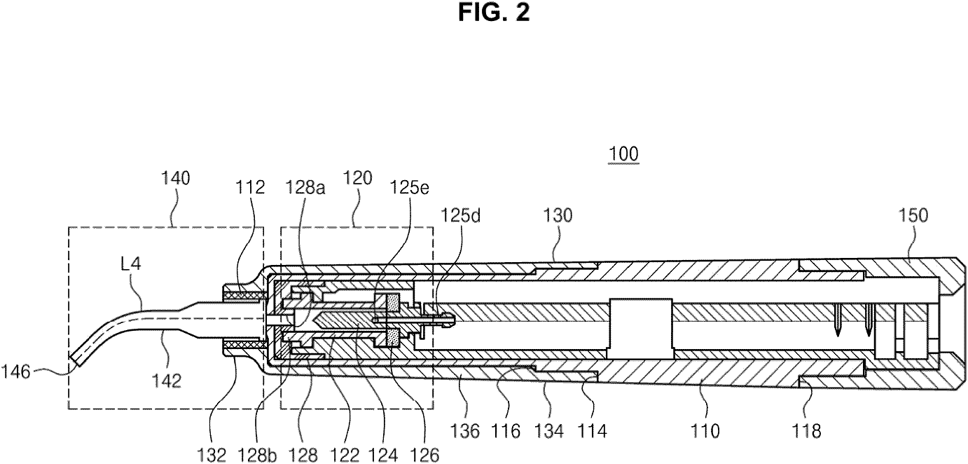

FIG. 2 is a perspective view of the handpiece that constitutes the dental disease treating device according to an embodiment of the present disclosure. Referring to FIG. 2, the handpiece 100 may include a handpiece body 110, a plasma generating part 120, a grip part 130, and a tip part 140.

The handpiece body 110 may have a size and a shape, by which a user easily grips it. The handpiece body 110 may have a substantially cylindrical shape. A space part, in which the plasma generating part 120 may be mounted, may be provided in an interior of the handpiece body 110.

The handpiece body 110 may be formed of a dielectric material having an insulating property. In an embodiment, the handpiece body 110 may be formed of a heat-resistant plastic or ceramic material having an excellent thermal conductivity, a heat resistance, and a corrosion resistance.

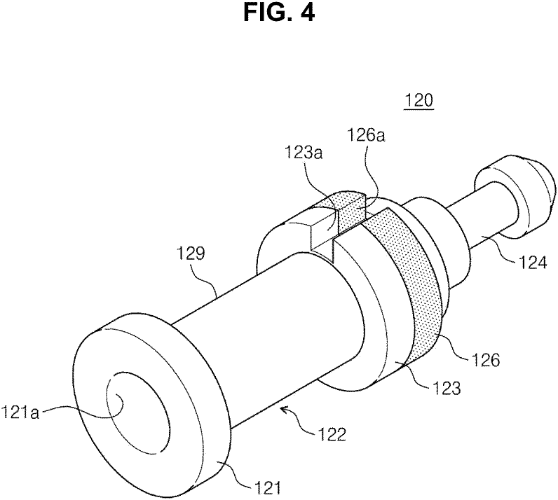

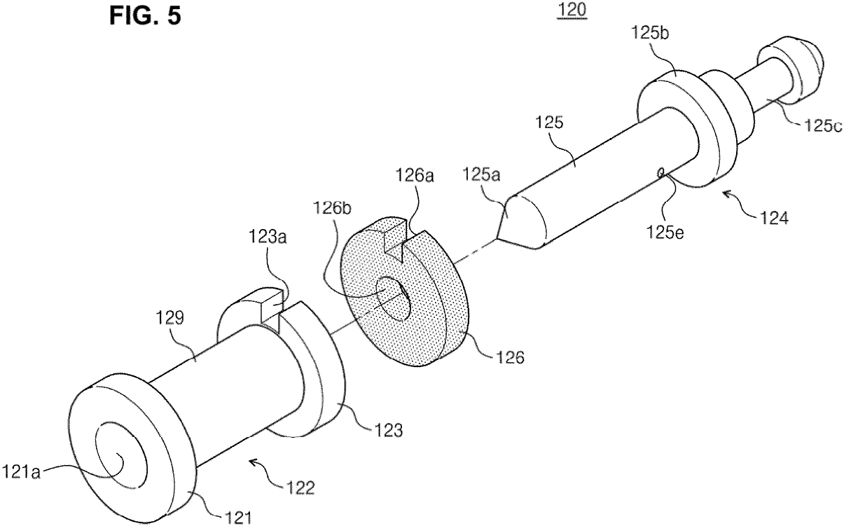

The plasma generating part 120 may be mounted in an interior of the handpiece body 110. The plasma generating part 120 may generate plasma by exciting the gas supplied through the supply line 200. The plasma generating part 120 will be described below with reference to FIGS. 4 and 5.

The grip part 130 may be mounted in an interior of the handpiece body 110. The grip part 130 may be coupled to the handpiece body 110 to be separable. Accordingly, the grip part 130 may be separated from the handpiece body 110 to separately perform treatments, such as washing and sterilization.

Similarly to the handpiece body 110, the grip part 130 may be formed of a dielectric material having an insulating property, and for example, may be formed of a heat-resistant plastic or ceramic material having an excellent thermal conductivity, a heat resistance, and a corrosion resistance.

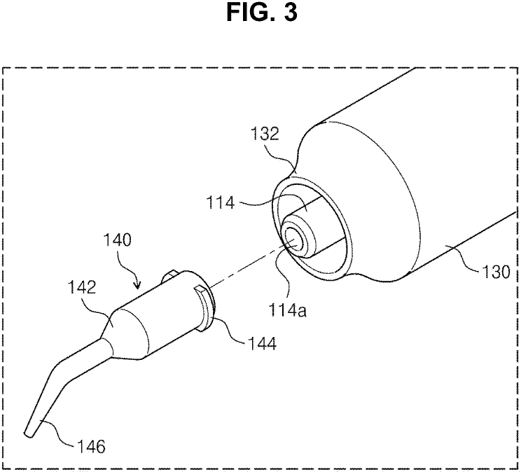

FIG. 3 is a view illustrating an attachment/detachment structure of the tip part that constitutes the dental disease treating device according to an embodiment of the present disclosure. Referring to FIGS. 2 and 3, the tip part 140 may be detachably coupled to an end nozzle 114 of the handpiece body 110 corresponding to an end of the plasma generating part 120 such that the plasma generated by the plasma generating part 120 is irradiated to a location portion. A Luer connector structure may be provided to be easily replaced and used.

The end nozzle 114 of the handpiece body 110 may be formed in an interior of a tip coupling part 112 provided at an end of the handpiece body 110. The tip coupling part 112 may be provided at an inner diameter portion of a tip end 132 of the grip part 130. A passage 114a, through which the plasma generated by the plasma generating part 120 may be delivered, may be formed in the end nozzle 114 of the handpiece body 110.

The tip part 140 may include a cylindrical tip body 142, a plasma delivery pipe 146 communicated with one side of the tip body 142 and having a cylindrical shape that is thinner than the tip body 142, and a coupling hole 144 formed on an opposite side of the tip body 142 to be coupled to the end nozzle 114 of the handpiece body 110.

The tip part 140 may function to inhibit generation of ozone by preventing the plasma from being exposed to the air while guiding the plasma into the oral cavity of a human body. That is, the tip part 140 may be mounted to inhibit generation of ozone by preventing the ambient air from penetrating into the plasma generating part 120 in the interior of the handpiece 100. The plasma delivery pipe 146 of the tip part 140 may have a deflected shape (a curved shape) to facilitate a treatment, for example, of a periodontitis or a decayed tooth in an interior of the oral cavity.

FIG. 4 is a perspective view of the plasma generating part that constitutes the dental disease treating device according to an embodiment of the present disclosure. FIG. 5 is an exploded perspective view of the plasma generating part that constitutes the dental disease treating device according to an embodiment of the present disclosure. Referring to FIGS. 2, 4, and 5, the plasma generating part 120 may include a body 122, an inner electrode 124, an elastic pad 126, a support 128, and an outer electrode 129.

The body 122 may have a cylindrical shape having an interior space, into which the inner electrode 124 is inserted. The body 122 may be formed of a dielectric material having an insulating property, and for example, may be formed of a heat-resistant plastic or ceramic material having an excellent thermal conductivity, a heat resistance, and a corrosion resistance. A first coupling part 121 having a first opening 121a may be formed at one end of the body 122. A second coupling part 123 having a second opening 123a may be formed at opposite end of the body 122.

A coupling recess 123a to be coupled to the body 122 may be formed in the second coupling part 123. The coupling recess 123a may be coupled to a coupling boss (reference numeral omitted) formed on an inner surface of the handpiece body 110.

The inner electrode 124 may include an electrode body 125 having a substantially cylindrical shape. A flange part 125b, and a gas introduction pipe 125c including a first passage 125d, through which the gas for generating the plasma is introduced, may be formed at a rear end of the electrode body 125. A tip end 125a of the electrode body 125 may be provided in a conical shape.

The tip end 125a of the inner electrode 124 may have a sharp tip end to increase a plasma density by reinforcing an electric field due to an electric charge concentration phenomenon, and may decrease a discharge initiation voltage that is a voltage, at which the plasma starts to be generated. Furthermore, due to a tapered shape of the tip end, an area, by which the plasma may be generated, may become wider, and thus an amount of the generated plasma may be increased.

The inner electrode 124 may be formed of an electrically conductive material, and for example, may be formed of chrome, stainless steel, nickel, copper, brass, vanadium, cobalt, platinum, gold, silver, and an alloy thereof. The inner electrode 124 may be grounded such that an electric shock may be prevented even though water for washing spit or an affected area of a patient is introduced into the plasma generating part 120 in the interior of the handpiece 100.

The first passage 125d of the gas introduction pipe 125c may be communicated with an interior passage of the electrode body 125. A plurality of gas discharge holes 125e for radially discharging the gas introduced through the first passage 125d of the gas introduction pipe 125c may be formed in the electrode body 125 in a radial direction.

The elastic pad 126 functions to seal an aperture between the body 122 and the inner electrode 124 such that the gas is prevented from being discharged or introduced through the aperture between the body 122 and the inner electrode 124. The elastic pad 126 may be interposed between the second coupling part 123 of the body 122, and the flange part 125b of the inner electrode 124. The elastic pad 126 may be formed of an insulating rubber material.

A coupling recess 126a to be coupled to the elastic pad 126 may be formed in the elastic pad 126. The coupling recess 126a may be coupled to the coupling boss formed on the inner surface of the handpiece body 110. An insertion hole 126b, into which the inner electrode 124 is inserted, may be formed at a central portion of the elastic pad 126.

The support 128 may have a vent hole 128a for delivering the plasma generated by the plasma generating part 120 to the tip part 140. The support 128 may have an insertion groove 128b, into which the tip end of the plasma generating part 120 may be inserted.

The outer electrode 129 may be formed on an outer surface of the body 122, through metalizing. The outer electrode 129 formed through metalizing may be formed of an electrically conductive material, and for example, may be formed of chrome, stainless steel, nickel, copper, brass, vanadium, cobalt, platinum, gold, silver, and an alloy thereof.

When an AC high voltage is applied between the outer electrode 129 and the inner electrode 124, the plasma may be generated in the plasma generating space between an inner surface of the body 122 and the inner electrode 124. Electrons, ions, and various radicals are present in the plasma, and the elements may be irradiated to the periodontitis located between the teeth and the gum or teeth, through the tip part 140 detachably coupled to the front end of the handpiece 100 to inhibit inflammations.

The grip part 130 may include a grip part body 136. The grip part body 136 may be mounted on an outer surface of the handpiece body 110. An insertion part 134 inserted into stepped parts 114 and 116 formed on the outer surface of the handpiece body 110 may be formed at a rear end of the grip part body 136. A step 118, to which a cover 150 is coupled, may be formed at a rear end of the handpiece body 110.

It is preferable that the gas supplied to the plasma generating part 120 is an inert gas, such as helium, neon, argon, or nitrogen. As the inert gas flows to a plasma discharge gas, a danger of generation of an arc may be reduced by reducing a discharge voltage.

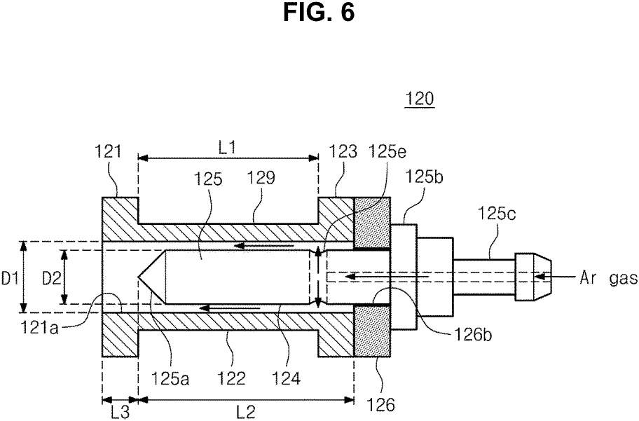

FIG. 6 is a cross-sectional view of the plasma generating part that constitutes the dental disease treating device according to an embodiment of the present disclosure. Arrows illustrated in FIG. 6 indicate flows of an argon gas for generating plasma. Hereinafter, referring to FIGS. 2 and 6, nozzle design parameters of the plasma generating part for efficiently reducing generated plasma and generated ozone will be described.

To obtain high-density plasma in a state, in which the tip part 140 is mounted, it is important to design various parameters, such as a length L1 of the outer electrode 129, a diameter D2 and a length L2 of the inner electrode 124, a diameter D1 of the interior passage of the body 122, and a distance between the outer electrode 129 and the inner electrode 124, in an optimum condition.

First, the length L1 of the outer electrode 129 may be designed to satisfy a parameter condition of 0.5 ≤L1/L2 ≤1 such that it has a ratio of not less than ½ and not more than 1 of the length L2 of the inner electrode 124, to increase a volume of the generated plasma.

Here, the length L2 of the inner electrode 124 means a distance from a location of the inner electrode 124, which corresponds to a front surface of the elastic pad 126, to a front end of the inner electrode 124, in a state in which the inner electrode 124 is inserted into the elastic pad 126. As an example, the length L2 of the inner electrode 124 may be designed to be 10 mm to 20 mm, and the length L1 of the outer electrode 129 may be designed to be 5 mm to 20 mm.

The diameter D2 of the inner electrode 124 may be designed to satisfy a parameter condition of 0.5 ≤D2/D1 ≤0.8 such that it has a ratio of not less than ½ and not more than ⅘ of the diameter D1 of the interior passage of the body 122, to increase a distance between the inner electrode 124 and the outer electrode 129 while increasing the volume of the generated plasma.

Accordingly, the high-density plasma may be generated by increasing the volume of the generated plasma and increasing a distance, by which the electrons may be accelerated. As an example, the diameter D2 of the inner electrode 124 may be designed to be 2 mm to 4 mm, and the diameter D1 of the interior passage of the body 122 may be designed to be 3 mm to 5 mm.

A distance between the plasma generating area and the front surface of the body 122 may be minimized. That is, a distance L3 between a front end of the inner electrode 124 and the front surface of the body 122 may be designed to satisfy a parameter condition of 0.5 ≤L3/D2 ≤1 such that it has a ratio of not more than ½ and not less than 1 of the diameter D2 of the inner electrode 124.

Accordingly, the electrons, the ions, and radicals generated in the plasma may be efficiently delivered to the oral cavity of the human body through the tip part 140. To achieve this, the distance L3 between the front end of the inner electrode 124 and the front surface of the body 122, for example, may be designed to be 1 mm to 4 mm.

Hereinafter, an experiment for verifying the performance of the dental disease treating device according to an embodiment of the present disclosure will be described. Antifungal performances of the dental disease treating device according to the embodiment of the present disclosure, for four species (S. mutans, P. gingivalis, C. albicans, and E. faecalis) in the oral cavity, and detailed operation mechanisms are investigated and revealed.

700 or more species of microorganisms are present in the oral cavity of the human body, and constitute one of the most complex microbial communities in the human body. Most of the microorganisms in the oral cavity are present in a form of harmless commensal floras, but some of them are directly and indirectly associated with generation of dental diseases, such as decayed teeth, periodontal diseases, or oral cancers.

For example, Streptococcus mutans generate acids on surfaces of teeth by using carbohydrate, and corrode the teeth and cause decayed teeth. Porphyromonas gingivalis is one of main etiological agents, which is observed in subgingival plaques of about 86% of periodontitis patients and causes periodontitis reactions.

Enterococcus faecalis is an anaerobic gram-positive coccus, and is a representative pathogenic bacterium that is normally a normal commensal flora but is discovered in about 90% of post-endodontic therapy pain and infection cases. Candida albicans that is a fungal bacterium is a harmless bacterium that is observed at a possibility of not less than 80% in the oral cavity of a healthy person, but growths thereof discriminatively increase in a specific environment to cause oral candidosis.

Accordingly, it is essential to effectively remove pathogenic microorganisms for a successful practice in a process of treating pathogenic dental diseases, such as decayed teeth, periodontitis, and the like. In most of the dental practices, a method for minimizing secondary infections by physically removing tissues infected by pathogenic bacteria and then removing residual pathogenic bacteria by using antibiotics and antifungal agents is mainly used, but some of the pathogenic bacteria in the oral cavity develop medical tolerances and cause side effects against some medicines. Accordingly, a plasma treatment device for an oral cavity that may perish pathogenic bacteria more effectively by minimizing use of the medicines is necessary.

To use cold plasma in a dental treatment, two big problems have to be overcome. That is, an amount of generated ozone that may be additionally generated when the plasma is generated and may cause severe damage to a respiratory system has to be minimized, and cold plasma that does not cause thermal damage to tissues of the oral cavity has to be generated. The microorganism perishing performance of the plasma appears as ions, electrons, reactive oxygen species, reactive nitrogen species, heat, UV light, which are agents generated in the process of generating the plasma, are complexly operated, the microorganism perishing performance may vary according to a plasma generation scheme.

Accordingly, even though a plasma generating technology that minimizes generation of ozone and heat for a dental operation, it is necessary to verify whether various oral pathogenic bacteria may be effectively sterilized, and a method for effectively treating cold plasma in various tissues of the oral cavity also has to be sought. Accordingly, a device for generating non-ozone cold plasma that inhibits generation of ozone at a low temperature has been developed.

The dental disease treating device developed according to the embodiment of the present disclosure was designed such that a tip part may be mounted for various dental operations, and changes in sterilization performances for four species of oral microorganisms (S. mutans, E. faecalis, P. gingivalis, and C. albicans) according to whether the tip part was mounted will be discussed. Furthermore, a dental application possibility will be discussed in detail by performing various studies for investigating and revealing a sterilization mechanism of the dental disease treating device.

A handpiece including an inner electrode of stainless steel and an outer electrode that surrounds an outer surface of a ceramic nozzle was manufactured for generating plasma. A Luer lock type coupling part was provided at an end of a plasma generating nozzle of a handpiece such that various tip parts may be attached to and mounted on the coupling part. Plasma was generated by causing an argon gas to flow between the inner electrode and the ceramic nozzle and applying an output voltage of 3 kVpp having a frequency of 20 kHz to opposite electrodes of the inner electrode and the outer electrode. Then, a flow rate of the argon gas that flows through the handpiece was adjusted by using an adjustment knob, by identifying a flow rate of the argon gas by using a gas flow rate controller.

Candida Albicans bacteria that were known as oral mucous membrane causasive organisms were used to measure an antifungal effect against the oral microorganisms of the plasma. The bacteria were used in experiments after being subcultivated to increase activity, and were cultivated in culture media of YM AGAR (DIFCO 0712) in a cultivator of a condition of 37° C. A Griess reagent kit product for measuring NO was purchased at Thermo fisher to be used, and an Amplex red hydrogen peroxide/peroxidase assay kit product was purchased at Invitrogen Co. (Carlsbad, CA) to be used.

To measure an antifungal performance of plasma for experimental floras, bacteria were painted on a YM solid culture medium at a concentration of 108 CFU/ml. TOP treatments were performed in unit of one hour at 2 slm in a condition, in which no tip was mounted, TOP treatments were performed in unit of one hour at 1 slm when a tip was mounted, and a diameter of a BRIC generated after bacteria were cultivated for 16 hours in a bacterium cultivation condition was measured.

To identify an influence of the TOP treatment on the growth of experimental floras, bacteria were divided to twenty four well plates by 10 µl and 990 µl of a liquid cultivation culture media ware added and prepared. A capillary tip was mounted on the TOP, and was treated at 1 slm for the time periods of 1 minute, 3 minutes, and 5 minutes, and then was cultivated for 5 hours and 24 hours in a cultivator. The cultivated bacteria were painted on a solid culture medium after being diluted to be 107 CFU/ml, and were cultivated again in a cultivator of 37° C. for sixteen hours, and the number of generated colonies were counted and analyzed.

To identify a change in a concentration of H₂O₂ due to the TOP treatment in the liquid cultivation culture media, an experiment was performed by using an Amplex Red hydrogen peroxide/peroxidase assay kit. The TOP was treated by the liquid culture medium to prepare a sample, 100 µ of Amplex Red reagent and 0.2 U/mL of HRP were mixed, and the same amounts were taken in ninety six well plates for a reaction. The reaction was treated for 30 minutes at a room temperature while light was shielded, and then absorbance was measured at 560 nm by using a microplate reader.

Furthermore, to measure a change in a concentration of a nitric oxide (NO) due to the TOP treatment in the liquid cultivation culture medium, a Griess assay method was used after the TOP treatment was performed on the culture medium. The prepared sample was treated by a reagent solution mixture and absorbance was measured at 548 mm with the microplate reader. All values of the experimental result were statistically processed by using an SPSS program. The statistical similarity of a control group and a test group was calculated with a t-test. An amount of the generated ozone was measured in unit of one minute of suction at a distance of 1 cm for 10 minutes by using APOA-360CE equipment of HORIBA Company.

As described above, to apply plasma to the oral cavity that was a respiratory system, it was essential to develop a plasma generating device that may minimize generation of ozone. Accordingly, a device was developed to use a high-concentration argon gas as a medium gas for generating plasma and to minimize contact with oxygen that is essential to form ozone around a plasma plume formed in the device. The device used for the experiment of the present disclosure had one cold plasma module, and was developed such that various tip parts may be mounted on a distal end of the plasma generating part.

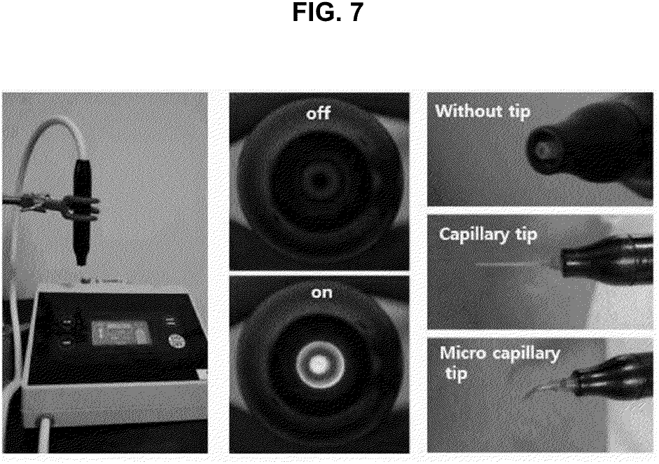

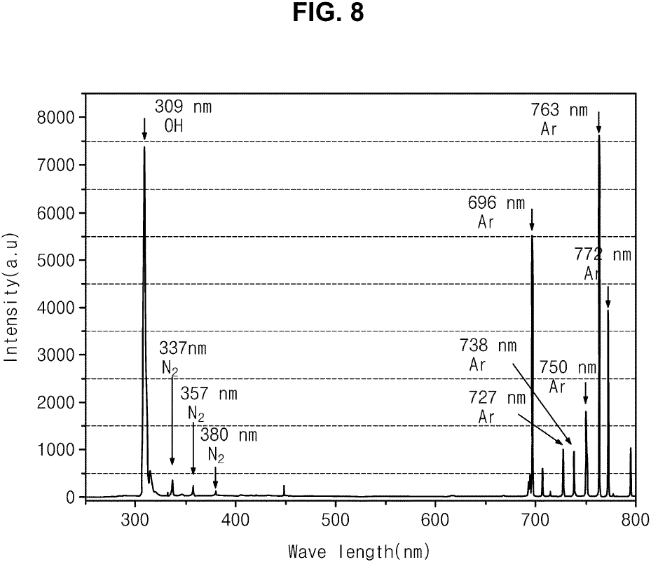

FIG. 7 is a picture depicting the dental disease treating device according to an embodiment of the present disclosure. FIG. 8 is a graph illustrating plasma active species elements generated by the dental plasma generating device, on which no tip was mounted. To analyze chemical constituent factors of the plasma generated by the dental plasma generating device, the plasma active species elements were measured in a state, in which the tip was not mounted, by using optical emission spectroscopy.

Referring to FIG. 8, it may be identified that the plasma generating device used for the experiment includes high OH radicals, low excited N₂, and high ionized argon, which are chemical features of the argon plasma. A change in the generated ozone according to whether the tip was mounted was identified to identify whether ozone was generated, which is one of the important purposes in developing the dental disease treating device.

Table 1

| 0₃ level (ppm) | w/o tip | with tip |
|---|---|---|
| Background | 0.006 | nd |
| 1 min | 0.020 | 0.001 |
| 2 min | 0.007 | nd |
| 3 min | 0.008 | 0.001 |
| 4 min | 0.009 | 0.002 |
| 5 min | 0.011 | 0.002 |
| 6 min | 0.011 | 0.003 |
| 7 min | 0.011 | 0.003 |
| 8 min | 0.012 | 0.003 |
| 9 min | 0.012 | 0.003 |
| 10 min | 0.012 | 0.003 |
| Maximum | 0.020 | 0.003 |
| Average | 0.011 | 0.002 |

It was identified from Table 1 that a maximum concentration of ozone was 0.020 ppm and an average concentration of ozone was 0.011 ppm in a result of the measurements for 10 minutes in a state (w/o tip), in which no tip was mounted, and a maximum concentration of ozone was 0.003 ppm and an average concentration of ozone was 0.002 ppm, which showed that a rate of generation of ozone was significantly decreased. To discuss an immediate surface sterilization performance for oral microorganisms of the plasma generated by the dental disease treating device developed through the experiment of the present disclosure, first, the sterilization performances were discussed for the bacteria after the four species of S. mutans, E. faecalis, C. albicans, P. gingivalis were treated for 1, 3, and 5 minutes.

Table 2

| TOP treatment | Diameter of inhibition zone(mm) | | |
|---|---|---|---|
| | 1 min | 3 min | 5 min |
| S. mutans | spot | 9 | 11 |
| E. faecalis | spot | 10 | 11 |
| C. albicans | 2 | 8 | 9 |
| P. gingivalis | - | 5 | 10 |

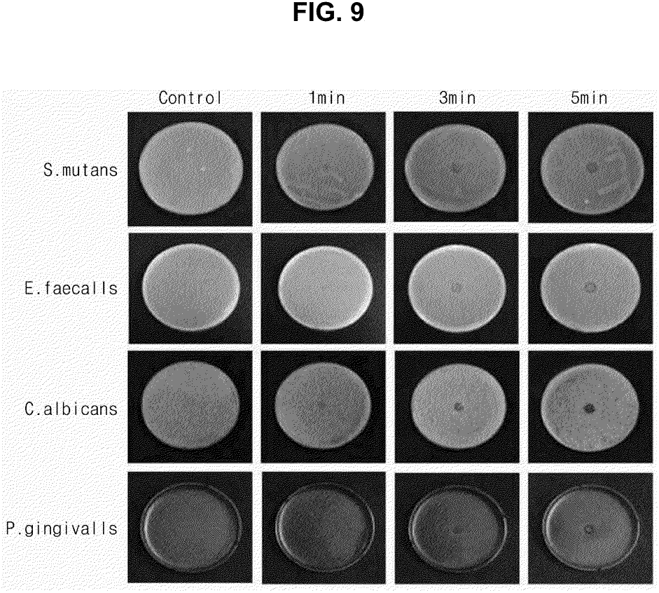

FIG. 9 is a picture depicting a result obtained by performing surface sterilizing treatments on four species of microorganisms by using a plasma device, on which no tip was mounted. Table 2 is a result that represents a size of the BRIC generated when surface sterilizing treatments are performed on four species of microorganisms by using the plasma device, on which no tip was mounted. Referring to FIG. 9 and Table 2, a zone, in which growth of microorganisms was inhibited, was dimly observed when S. mutans were treated for one minute by using cold plasma, and it could be identified that a BRIC of a diameter of 9 mm was formed when they were treated for three minutes and a BRIC of a diameter of 11 mm was formed when they were treated for five minutes. A spotted BRIC was formed when the cold plasma was treated for one minute for E. feacalis, a BRIC of a diameter of 10 mm was formed when it was treated for three minutes, and a BRIC of a diameter of 11 mm was formed when it was treated for five minutes. For C. albicans, a BRIC of a diameter of 2 mm was formed even when the cold plasma was treated only for one minute, a BRIC of a diameter of 8 mm was formed when it was treated for three minutes, and a BRIC of a diameter of 9 mm was formed when it was treated for five minutes. It could be identified that no particular perishing performance was observed when the cold plasma was treated for one minute for P. gingvalis, but a BRIC of a diameter of 5 mm was formed when it was treated for three minutes and a BRIC of a diameter of 10 mm was formed when it was treated for five minutes.

To discuss whether a surface sterilization performance of the cold plasma was maintained as it was even when a tip was mounted, a reaction to the bacterium perishing performance using C. albicans was discussed. FIG. 10 is a picture depicting a result obtained by performing surface sterilizing treatments on microorganisms by using a cold plasma device, on which no tip was mounted, and a cold plasma device, on which a tip was mounted. Table. 3 represents sizes of BRICs when surface sterilizing treatments were performed on microorganisms by using the cold plasma device, on which no tip was mounted, and the cold plasma device, on which a tip was mounted.

Table 3

| | | | |
|---|---|---|---|
| Tip -/+ | Diameter of inhibitions zone(mm) | | |
| | 1 min | 3 min | 5 min |
| Without Tip | 2 | 8 | 9 |
| Capillary Tip | - | - | - |
| Micro Capillary Tip | - | - | - |

As illustrated in FIG. 10 and Table 3, it could be observed that BRICs of diameters of 2 mm, 8 mm, and 9 mm were formed when the cold plasma was treated for one minute, three minutes, and five minutes with no tip. Meanwhile, it could be identified that a clear BRIC was not formed even when the cold plasma was treated for C. albicans for five minutes that was a maximum treatment time when the cold plasma was treated after two kinds of capillary tips were mounted. To objectively numericalize the sterilization performances of the cold plasma for four oral microorganisms, an influence of the cold plasma on growth of oral microorganisms that were being cultivated in a liquid culture medium was discussed. After the cold plasma was treated for one, three, and five minutes in liquid culture media, in which S. mutans, E. faecalis, C. albicans, P. gingivalis were inoculated, respectively, they were cultivated in the corresponding culture media for five hours or twenty four hours.

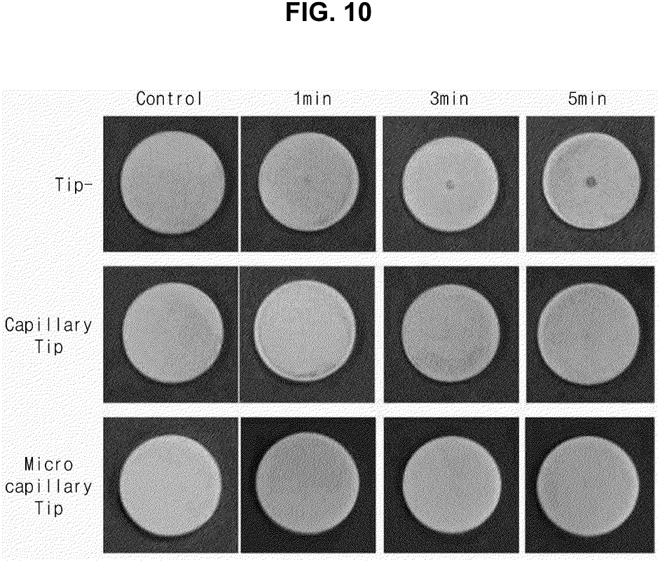

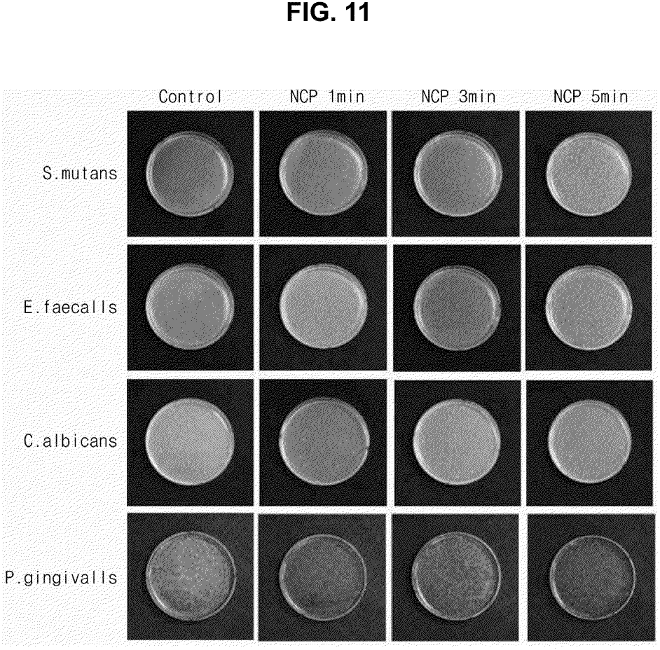

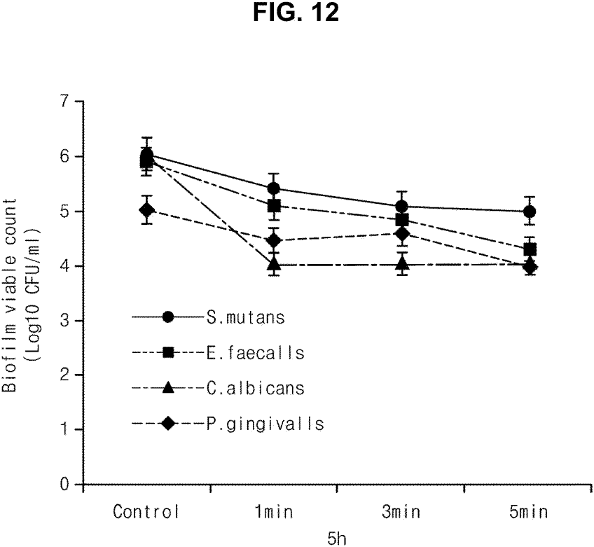

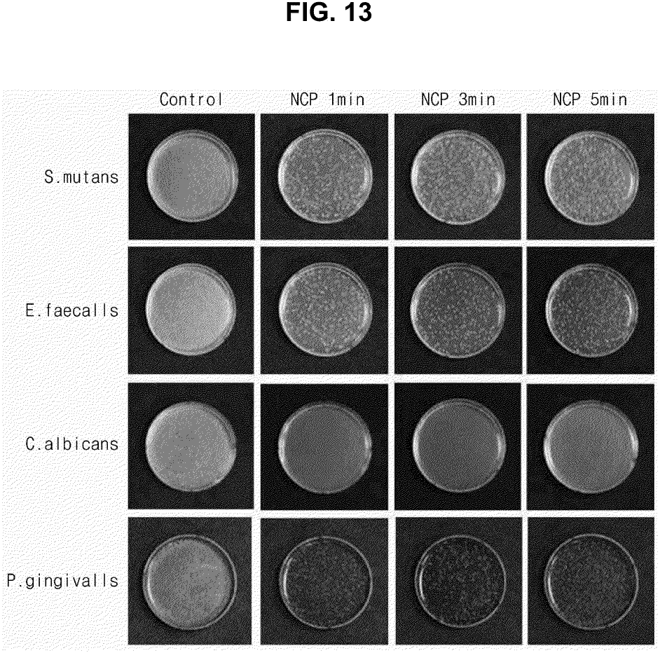

A bacterium growth inhibiting performance analysis (CFU) value was measured by diluting cultivated bacteria, inoculating the bacteria into a slid culture medium, and counting colonies generated after sixteen hours. FIG. 11 is a picture depicting a result obtained by cultivating microorganisms for five hours after the cold plasma treatment of the microorganisms. FIG. 12 is a graph depicting values obtained by analyzing bacterium growth inhibiting performances when microorganisms are cultivated for five hours after the cold plasma treatment of the microorganisms. FIG. 13 is a picture depicting a result obtained by cultivating microorganisms for twenty four hours after the cold plasma treatment of the microorganisms. FIG. 14 is a picture depicting a result obtained by cultivating microorganisms for twenty four hours after the cold plasma treatment of the microorganisms.

Referring to FIGS. 11 to 14, when the cold plasma was treated on the liquid culture medium, into which S. mutans was inoculated, a growth inhibiting performance of 0. 5 log was observed when it was treated for one minute, and growth inhibiting performances of about 1 log were observed when it was treated for three minutes and five minutes, at a time point, at which five hours elapsed, whereas decreased performances of about 5.5 log were observed in the samples, which were treated for about one minute, three minutes, and five minutes when it was treated for twenty four hours. It was identified that when the cold plasma was treated for one, three, and five minutes for E. feacalis, growths were inhibited by 1 log, 1.2 log, and 1.8 log at a time point, at which five hours elapsed, and about 5.5 log were decreased in all the samples that were treated for one, three, and five minutes at a time point, at which twenty four hours elapsed.

For C. albicans, decreases of the number of cells of about 2 log were measured in all the samples that were treated for one minute, three minutes, and five minutes when five hours elapsed after the cold plasma was treated, and growth inhibiting phenomena of 5.5 log or more were observed in all of the samples that were treated for one minute, three minutes, and five minutes when twenty four hours elapsed. Finally, for P. gingivalis, growth inhibits of about 0.5 log were observed when it was treated for one minute, 0.4 log for three minutes, and about 1 log for five minutes, at a time point, at five hours elapsed after the cold plasma was treated, and growth inhibiting phenomena of about 5.4 log were observed in all of the samples, for which the cold plasma was treated for one, three, and five minutes after the cold plasma was treated.

It was discussed how the growth inhibiting performance for the oral microorganisms in the liquid culture medium of the NCP that was identified above was changed when a tip was mounted. FIG. 15 is a graph depicting a result obtained by comparing growth inhibiting performances by cold plasma treatments of four species of microorganisms according to whether a tip was mounted.

It was discussed how the growth inhibiting performance for the oral microorganisms in the liquid culture medium of the NCP that was identified above was changed when a tip was mounted. A growth inhibiting performance of about 6 log was observed in a sample, in which the cold plasma was treated for E. faecalis, when a capillary tip was mounted, whereas a growth inhibiting performance of about 5 log was observed in a sample, in which no tip was mounted, and a sample, in which a micro-capillary tip was mounted and the cold plasma was treated.

Growth inhibiting performances of 6 log for C. albicans were observed in both of a sample, in which no tip was mounted and the cold plasma was treated, and a sample, in which a capillary tip was mounted and the cold plasma was treated, whereas a growth inhibiting performance of 5 log was observed in a sample, in which a micro-capillary tip was mounted and the cold plasma was treated.

For P. gingivalis, growth inhibits of about 6 log were observed when two kinds of tips were mounted, in which the cold plasma was treated for three minutes or more, and a growth inhibiting performance of about 8 log was observed when no tip was mounted. When the cold plasma was treated for one minute, a growth inhibiting phenomenon of about 5 log was observed in the sample, in which the micro-capillary tip was mounted, and showed a low growth inhibiting performance of about 1 log as compared with other samples.

To identify through which operation mechanism an oral microorganism growth inhibiting phenomenon appeared due to the NCP in the liquid culture medium, first, changes in active species generated in the liquid culture medium when the cold plasma was treated were discussed. FIG. 16 is a graph depicting changes in a concentration of H₂O₂ of a culture medium when a cold plasma treatment is performed on a microorganism culture medium. Referring to FIG. 16, when the cold plasma was treated for 0.5 minutes, one minute, three minutes, and five minutes in the microorganism culture medium, a phenomenon, in which a concentration of H₂O₂ of the culture medium was changed to 15 µM, 22 µM, 95 µM, and 175 µM, respectively.

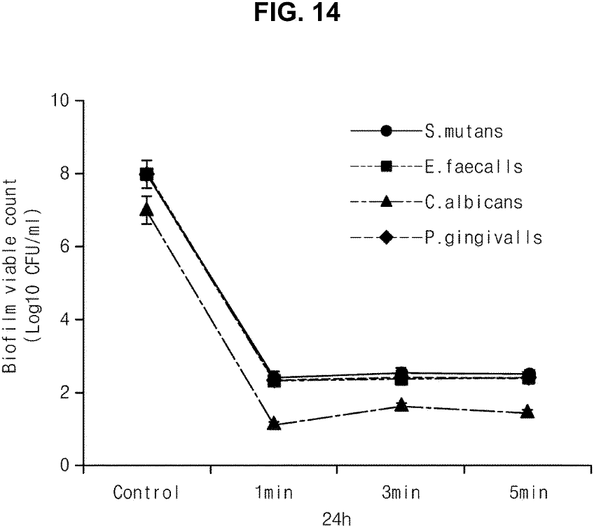

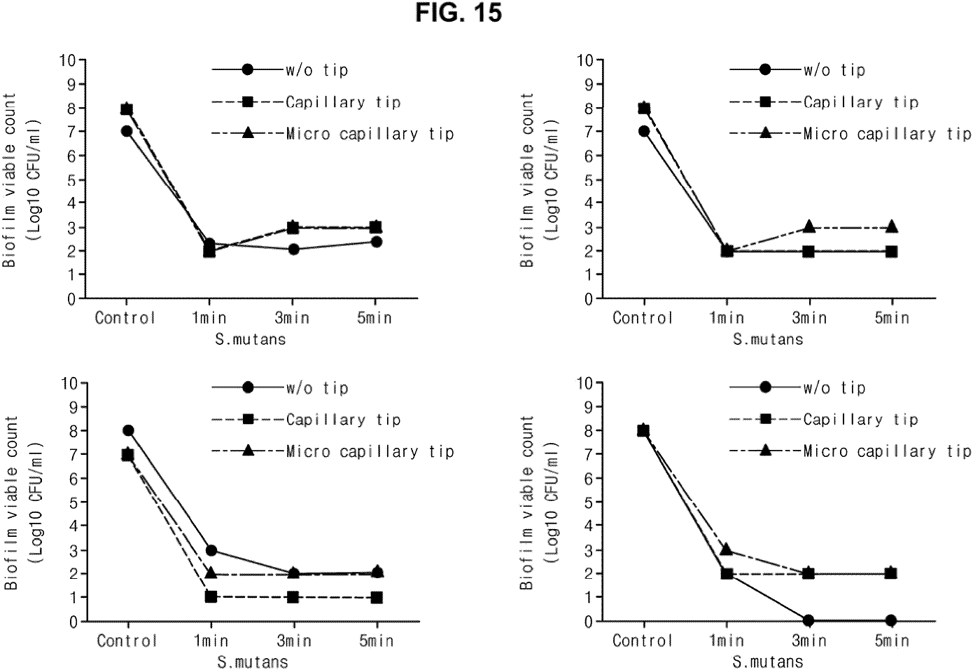

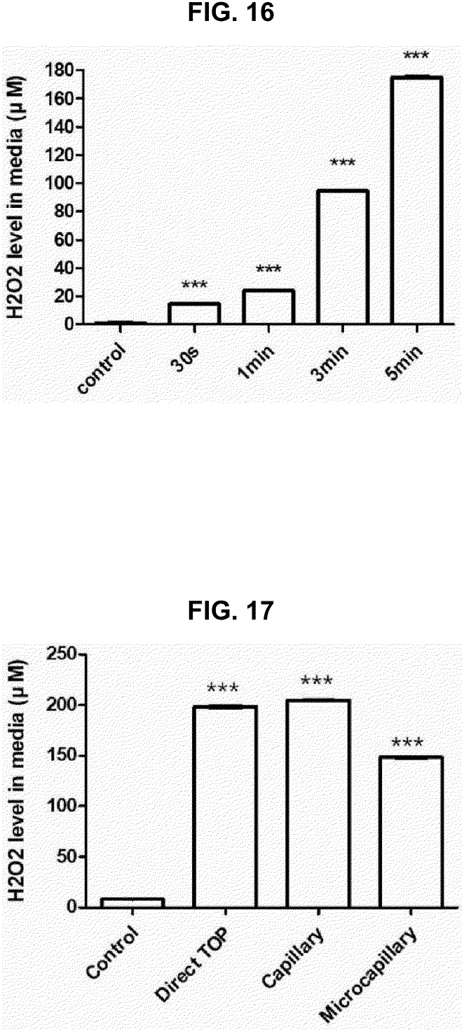

FIG. 17 is a graph depicting changes in a concentration of H₂O₂ of a culture medium during a cold plasma treatment according to whether a tip is mounted. When the capillary tip was mounted, a concentration of 205 µM that was rather higher than a concentration (180 µM) in the culture medium obtained by directly treating the cold plasma while no tip is mounted was observed, whereas a relatively low concentration of 148 µM was observed in the culture medium obtained by mounting a micro-capillary tip and treating the cold plasma.

Meanwhile, to discuss changes reactive nitrogen species generated in the liquid culture medium when the cold plasma was treated, changes in a concentration of a nitrite (N0₂⁻) of the liquid culture medium were discussed. FIG. 18 is a graph depicting changes in a concentration of a nitrite of a liquid culture medium during a cold plasma treatment. When the cold plasma was treated for one minute, three minutes, and five minutes in the liquid culture medium, a phenomenon, in which a concentration of the nitrite of 1.66 µM was gently increased to 1.72 µM, 1.8 µM, and 1.87 µM, respectively, was observed.

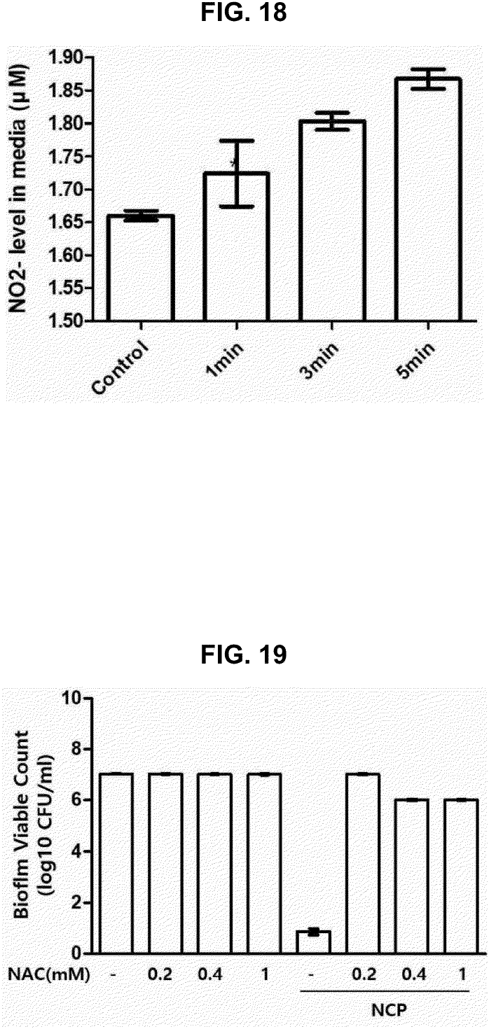

To identify whether the change in the concentration of H₂O₂ of the liquid culture medium by the cold plasma played an important role in the oral microorganism growth inhibiting phenomenon by the cold plasma, a study, to which N-acetylcysteine (NAC) that is a H₂O₂ scavenger was applied, was performed. FIG. 19 is a view illustrating changes in inhibition of growth of microorganisms according to a cold plasma treatment and application of N-acetylcysteine. It could be identified that growth of cells was not greatly influenced when 0.2 mM, 0.4 mM, and 1 mM of N-acetylcysteine were treated on S. mutans inoculated into the liquid culture medium, whereas growth of the cells was inhibited by about 6.8 log when the cold plasma was treated for five minutes. It could be identified that the growth inhibiting performance by the cold plasma was recovered when both the cold plasma and the N-acetylcysteine were treated at the same time.

The fact that no surface sterilizing performance of the cold plasma was observed in the solid culture medium when the tip was mounted means that a sterilization mechanism of the cold plasma in the solid culture medium and an operation mechanism in the liquid culture medium were different. A possibility of a sterilization force being decreased as a distance between the plasma generating part and the oral microorganisms becomes larger when the tip used in the experiment of the present disclosure is mounted was discussed.

Table 4

| Capillary Tip | Diameter of inhibitions z one(mm) | | |
|---|---|---|---|
| | 1 min | 3 min | 5 min |
| 1.5 cm | 3 | 5 | 7 |
| 2.0 cm | 2 | 4 | 7 |
| 2.5 cm | - | 3 | 6 |
| 3.5 cm | - | Spot | 4 |

FIG. 20 is a picture depicting a change in a surface sterilizing treatment for microorganisms according to a length of the capillary tip and a cold plasma treatment time period. FIG. 21 is an exemplary view of various capillary tips used in experiments. Table 4 represents a size of the BRIC when a surface sterilizing treatment was performed on microorganisms according to the length of the capillary tip and the cold plasma treatment time period. Referring to FIGS. 20 and 21, and Table 4, a BRIC of a diameter of 4 mm was formed when a tip of a length of 2 mm was mounted and the cold plasma was treated for five minutes, and a BRIC of a diameter of 6 mm was formed when a tip of 1 cm was mounted. It could be identified that BRICS of a diameter of 7 mm were formed in both cases when a tip of 0.5 cm was mounted and when a tip of 0 cm was mounted (when there is substantially no tip). It could be identified that a BRIC of a larger diameter was formed as a length of a mounted tip becomes smaller even when the cold plasma was treated for one minute and three minutes.

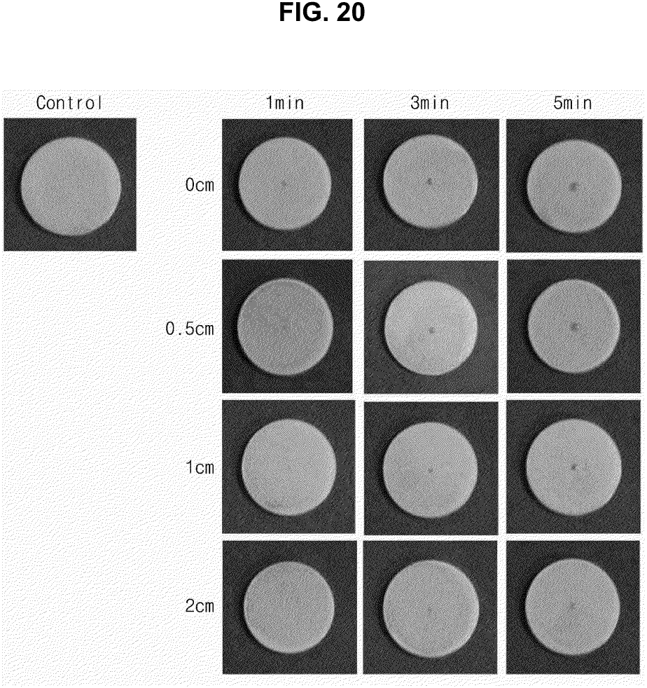

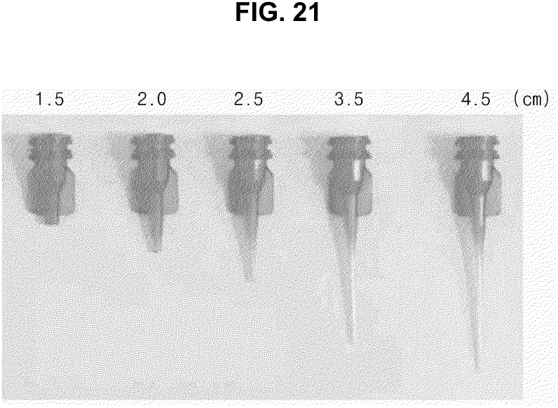

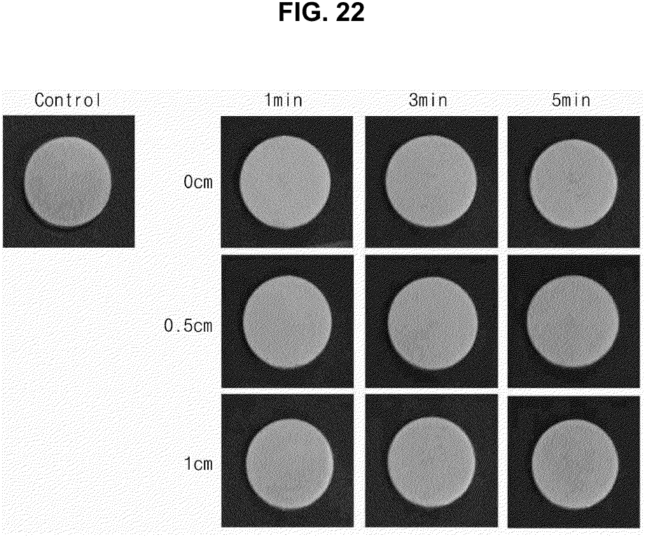

FIG. 22 is a picture depicting a change in a surface sterilizing treatment for microorganisms according to the length of the micro-capillary tip and the cold plasma treatment time period. FIG. 23 is an exemplary view of various micro-capillary tips used in experiments. Table 5 represents sizes of BRICs during a surface sterilizing treatment for microorganisms according to the length of the micro-capillary tip and the cold plasma treatment time period. Referring to FIGS. 22 and 23, and Table 5, it could be identified in a study result obtained by observing changes in a surface sterilization force of the cold plasma according to a change in the length of the micro-capillary tip that no BRIC was formed even when the length of the tip is as short as 0.5 cm and 0 cm.

Table 5

| Cm | Diameter of inhibitions zone(mm) | | |
|---|---|---|---|
| | 1 min | 3 min | 5 min |
| 3 | - | - | - |
| 3.5 | - | - | - |
| 4 | - | - | - |

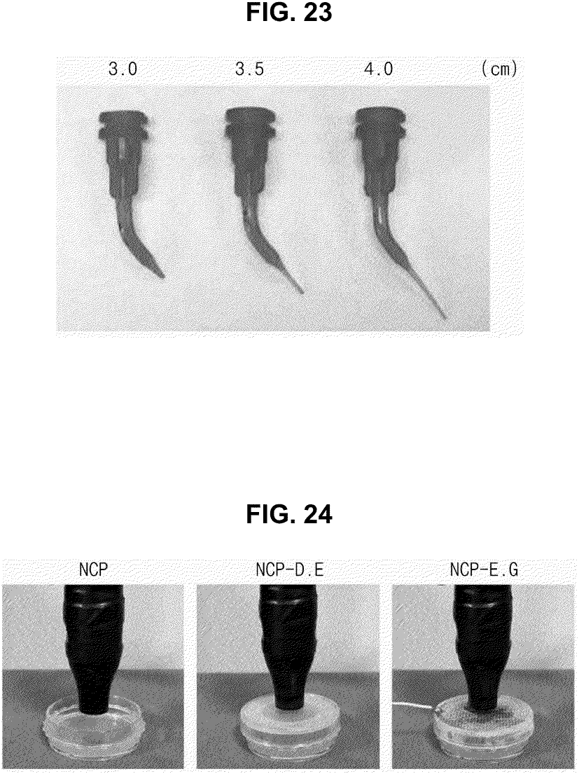

To investigate and reveal which active factor of the plasma played an important role in the surface sterilization performance of the cold plasma, a change in the surface sterilization performance of the cold plasma was discussed after two kinds of meshes were installed between the cold plasma treating device and a solid culture medium, into which C. albicans was inoculated. FIG. 24 is a picture depicting an experiment through observation of surface sterilizing performances of cold plasma by installing a mesh between the cold plasma treating device and the microorganism solid-culture medium. FIG. 25 is a picture depicting an experimental result through observation of surface sterilizing performances of cold plasma by installing a mesh between the cold plasma treating device and the microorganism solid-culture medium. It could be identified that a microorganism surface sterilizing performance that was similar to that of a case, in which simply the cold plasma (NCP) was treated, when the cold plasma was treated on microorganisms via a non-electric mesh was shown (NCP-D.E). Meanwhile, it could be identified that a size of the BRIC generated after the cold plasma was treated for the same period of time is significantly reduced when a grounded electric mesh is installed between the microorganisms and the cold plasma device (NCP-E.G).

To discuss whether surface sterilization performances of the cold plasma for the types of the meshes were changed due to a change of generation of H₂O₂, the changes of H₂O₂ according to two types of meshes were discussed after two types of meshes were installed when the cold plasma was treated for five minutes in the liquid culture medium. FIG. 26 is a graph depicting the changes in the generated H₂O₂ according to the types of meshes during the cold plasma treatment. It could be identified that about 250 µM of H₂O₂ was generated in the culture medium when no mesh was installed and the cold plasma was directly treated in the liquid culture medium (a direct TOP) but about 150 µM of H₂O₂ was formed in the liquid culture media, on which the cold plasma was treated after the two types of meshes (E.G and D.E) were installed.

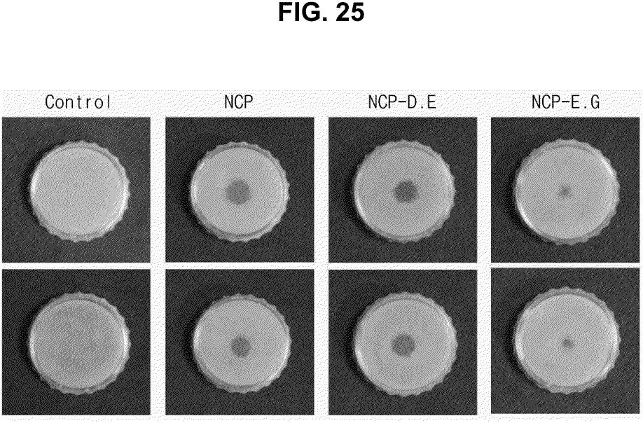

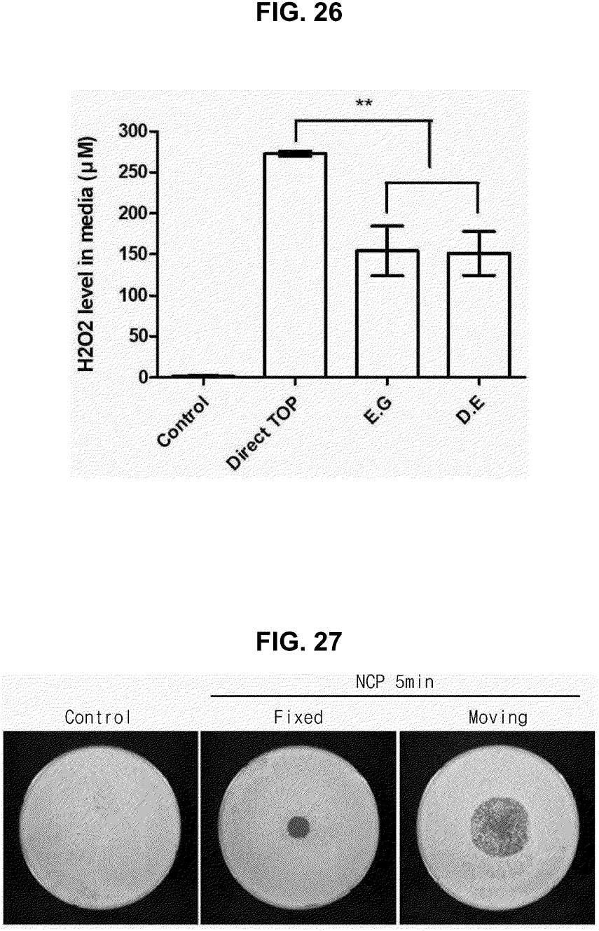

The result hinted that the surface sterilization performance of the cold plasma was due to electrons or electric charge particles that may be removed by the grounded electric mesh. A possibility of an effective range of the sterilization performance of the cold plasma becoming larger when the cold plasma was treated at various locations than while being fixed at one point for five minutes. FIG. 27 is a picture depicting a result through observation of the effective range of the sterilizing performances when the cold plasma treatment was made at a fixed location and at variable locations. It could be identified that a thick BRIC of a diameter of about 1 cm was formed when the cold plasma was treated at a fixed location whereas a rather wide but slightly thin BRIC was formed on a solid culture medium when the cold plasma was treated for the same period of time while being repeatedly moved in a circular shape of a diameter of 3 cm.

To apply the cold plasma to various dental practices, it is necessary to mount various shapes of tips on the cold plasma device and apply the surface sterilization performance to a treated portion. A sterilization performance for oral bacteria was discussed after various shapes of tips were mounted on the cold plasma device to secure a condition of the tips for maintaining the sterilization performance of the cold plasma. A change in the shape of the BRIC was discussed twenty four hours after various tips were mounted on the cold plasma device, and the cold plasma was treated for five minutes while a distance between the solid culture medium, into which C. albicans was inoculated, and an end of the tip was maintained at 10 mm.

Table 6

| Tip | Name | Pore size | Tip length (cm) |
|---|---|---|---|
| 1 | Endo-Eze ™ Tip | 22 gauge | 3.5 |
| 2 | Endo-Eze ™ Tip | 20 gauge | 3.5 |
| 3 | Endo-Eze ™ Tip | 18 gauge | 3.5 |
| 4 | Micro 20 ga Tip | 20 gauge | 2.9 |
| 5 | White Mac™ Tip | 12 gauge | 3 |
| 6 | White Mini™ Tip | 16 gauge | 2.9 |
| 7 | SST™ Surgical Suction Tip | 10 gauge | 4.8 |

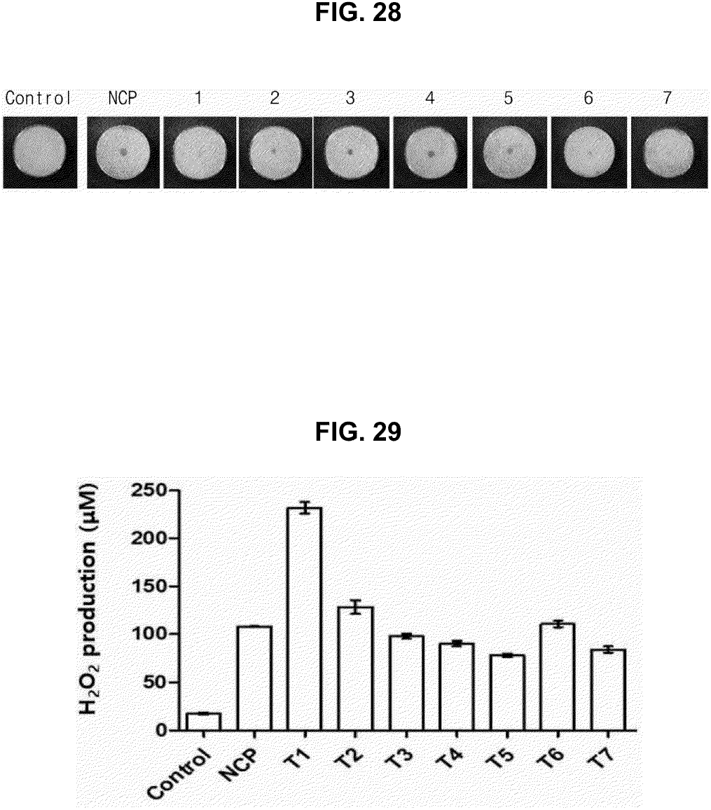

Table 6 summarizes lengths and inner diameters of various tips used in an experiment. FIG. 28 is a picture depicting changes in a shape of a BRIC according to changes of the length and the inner diameter of the tip during a cold plasma treatment. Table 7 represents the changes in a size of the BRIC according to the changes of the length and the inner diameter of the tip during the cold plasma treatment. FIG. 29 is a graph depicting changes in a concentration of H₂O₂ according to the length and the inner diameter of the tip during the cold plasma treatment.

Table 7

| Tip | | Diameter of inhibitions zone (mm) | | | | | | |
|---|---|---|---|---|---|---|---|---|
| | - | 1 | 2 | 3 | 4 | 5 | 6 | 7 |
| | 9 | 1.5 | 4 | 5.5 | 7.5 | 6 | 2 | |

Table 8

| | | | |
|---|---|---|---|
| Capillary Tip | Diameter of inhibitions zone (mm) | | |
| | 1 min | 3 min | 5 min |
| 0 cm | spot | 5 | 7 |
| 0.5 cm | - | 4 | 6 |
| 1 cm | - | 3 | 5 |
| 2 cm | - | spot | spot |

Table 9

| Capillary Tip | Diameter of inhibitions zone (mm) | | |
|---|---|---|---|
| | 1 min | 3 min | 5 min |
| 0 cm | 2 | 5 | 7 |
| 0.5 cm | spot | 4 | 5 |
| 1 cm | - | 4 | 4 |
| 2 cm | - | spot | 3 |

Table 10

| Capillary Tip | Diameter of inhibitions zone (mm) | | |
|---|---|---|---|
| | 1 min | 3 min | 5 min |
| 0 cm | 3 | 5 | 7 |
| 0.5 cm | 2 | 4 | 7 |
| 1 cm | - | 3 | 6 |
| 2 cm | - | spot | 4 |

Table 8 represents the changes in a size of the BRIC according to the changes of the length and the inner diameter of the tip during the cold plasma treatment of S. mutans microorganisms. Table 9 represents the changes in a size of the BRIC according to the changes of the length and the inner diameter of the tip during the cold plasma treatment of E. faecalis microorganisms. Table 10 represents the changes in a size of the BRIC according to the changes of the length and the inner diameter of the tip during the cold plasma treatment of C. albicans microorganisms. Referring to Tables 6 to 10, and FIGS. 28 and 29, it could be identified that, in spite that Nos. 1 to 3 tips had the same material and the same length, No. 1 tip did not form any BRIC whereas Nos. 2 and 3 tips formed BRICs that became wider as gauges of the tips became smaller. It could be identified that a BRIC was formed even when a bent tip of gauge 20 was used as in No. 4 tip, and a BRIC was formed when No. 5 tip having a larger diameter was mounted and the cold plasma was treated only in Nos. 5 and 6 tips that were manufactured of the same material and had only different diameter of ends of the tips. It could be identified through an experimental result of No. 7 tip that no BRIC was formed when the length of the tip was too long even when the tip had the largest end.

In spite of a strong sterilization performance of plasma, ozone that was additionally generated when the plasma was generated may be a big threat to a respiratory system that is directly connected to an oral cavity. When oxygen molecules collides with electrons having high energy during a process of generating plasma, the oxygen molecules are decomposed to singlet oxygen and then are bonded to nearby oxygen molecules and ozone may be generated. Accordingly, a plasma generating device was designed to have a structure, in which oxygen in air could be prevented from approaching a section, in which plasma was generated, by using argon as a medium gas to minimize ozone generated when the cold plasma was generated. Furthermore, the device was developed to have a structure, in which various tips could be mounted to effectively apply plasma to an oral cavity and teeth having complex structures.

As described above, it could be identified that the dental disease treating device designed according an embodiment of the present disclosure could effectively generate cold plasma and generate a large amount of OH radicals through a plasma chemical reaction that was unique to argon plasma when an OES analysis result was discussed. A study using a ultra-precision ozone measurement device was performed to identify whether the plasma generating device developed through the experiment of the present disclosure could inhibit generation of ozone, and as a result, when the cold plasma was generated for ten minutes by using the corresponding device, a maximum concentration of the generated ozone at a distance of 1 cm from a gas discharge hole was measured to be 0.02 ppm. The numerical value was measured to be 0.003 ppm when a tip was mounted on the corresponding device.

This means that ozone, a value of which was lower than 0.051 ppm (100 µg/m³) that was an ozone allowable value in air, which was described in Air Quality Guidelines provided by World Health Organization (WHO), by two times and a maximum of 10 times, and means that the present disclosure is a non-ozone plasma generating technology that did hardly generate ozone. Furthermore, a temperature of the plasma gas discharged from the corresponding device was measured to be 27.0° C. when no tip was mounted and to be 25.7° C. when a tip was mounted, and thus the plasma gas was cold plasma. In this way, the dental disease treating device according to the embodiment of the present disclosure could generate plasma, in which generation of ozone was maximally inhibited, while maintaining a low temperature that did not cause a thermal damage to tissues of an oral cavity to safely use various medical performances of the cold plasma in the oral cavity.

To discuss whether the cold plasma that hardly generates ozone and is being at a low temperature effectively sterilized various oral pathogens, sterilization performances of the NCP using S. mutans that are cavity bacteria, E. faecalis that cause periodontitis, P. gingivalis that cause periodontitis, and C. albicans that are a main cause of oral candidosis were discussed. It could be identified that the cold plasma immediately perished four species of bacteria that are in the solid culture medium. This may be a very particular result in that the oral microorganisms were exposed only to after-glows while not directly contact the plasma glow, and in that temperature was maintained at 27° C. or less while a concentration of O₃ was maintained at a reference value or less in the corresponding treatment condition.

A performance of sterilizing surfaces of oral microorganisms by the cold plasma was maintained even when the tip was mounted. When the bacterium perishing performance for S. mutans was performed by using two kinds of tips, it could be identified that the surface sterilizing performance for S. mutans disappeared when two kinds of tips were mounted. The phenomenon also appeared in three different oral microorganisms.

To numericalize the performance of sterilizing four species of oral microorganisms by the cold plasma, the performance of perishing bacteria of the oral microorganisms that were present in the liquid culture medium is examined. It could be identified that when the cold plasma was treated in the culture medium, in which four species of microorganisms were present, the microorganisms were additionally cultivated for five hours, and the colony forming unit was measured, a bacterium perishing performance of about 90% was observed only in the sample that was treated for five minutes for S. mutans, P. gingivalis, E. faecalis whereas about 2 log was decreased even when it was treated for one minute for C. albicans.

This means that C. albicans was effectively perished by the cold plasma as compared with the other three species of oral microorganisms. Meanwhile, when the cold plasma was treated, the bacteria were additionally cultivated in the liquid culture medium for twenty four hours, and a bacterium perishing performance of the cold plasma was observed, a bacterium sterilizing performance of 5 log or more was identified even through the cold plasma was treated only for one minute for the four species of oral microorganisms.

It was identified that the oral microorganism perishing performance in the liquid culture medium by the cold plasma was maintained even when the capillary tip and the micro-capillary tip were mounted. The result hints that the oral microorganism surface sterilizing operation in the solid culture medium by the cold plasma and the sterilization operation in the liquid culture medium by the cold plasma were made by different mechanisms.

It could be identified in a study result that investigated and revealed an action mechanism of various kinds of cold plasma tried in the experiments of the present disclosure that the oral microorganism perishing performance in the liquid culture medium by the cold plasma was caused by the phenomenon, in which an amount of H₂O₂ in the culture medium was increased through the treatment of the cold plasma. When the cold plasma was treated for the liquid culture medium, a concentration of hydrogen peroxide of the liquid culture medium was increased depending on a treatment time period of the cold plasma, and a level (200 µM) of H₂O₂ that was similar to that of the culture medium, for which simply the cold plasma was treated when the cold plasma was treated for five minutes after the capillary tip was mounted, whereas 150 µM of H₂O₂ was observed when the cold plasma was treated after the micro-capillary tip was mounted.

When the oral microorganism perishing performance in the liquid culture medium was discussed above, the bacterium perishing performances of 5 log or more appeared for the three species of bacteria even when the cold plasma was treated for one minute, and thus it may be determined that the bacterium perishing performance of 5 log or more was caused when a concentration of H₂O₂ in the liquid culture medium was increased to 20 µM or more by the cold plasma. Furthermore, because it was identified even in a study result using N-acetylcysteine that is a strong scavenger of H₂O₂ that the bacterium perishing performance of the cold plasma for S. mutans that was being grown was completely inhibited in the culture medium that was present at concentrations of N-acetylcysteine of 0.2, 0.4, and 1 mM, it could be identified that the oral bacterium perishing performance of the cold plasma in the liquid culture medium was due to H₂O₂.

It was determined that the cold plasma device developed according to the embodiment of the present disclosure increased a concentration of nitrite of the liquid culture medium very slightly but a mere change in a concentration of nitrous acid of the culture medium did not influence a great influence on the perishing performance of the cold plasma. A phenomenon, in which an amount of H₂O₂ that appeared in the culture medium when the cold plasma was treated was increased could be explained by an OES analysis result of the cold plasma.

In the OES analysis result of the plasma generating part of the cold plasma device, it could be identified that the largest amounts of OH radicals and excited argon were generated while the cold plasma was generated whereas the excited nitrogen was generated very weakly. Because the cold plasma used in the present disclosure hardly generated the excited N₂ systems, it is considered that the change in the concentration of the nitrite was very weak even when the cold plasma was treated for the liquid culture medium. Furthermore, it is determined that a large amount of OH radicals were generated by argon plasma and the concentration of H₂O₂ was abruptly increased when the treatment was made on the culture medium.

It was identified that the bacterium perishing performance in the solid culture medium by the cold plasma was achieved in a scheme that was independent from a H₂O₂ generation ability unlike the bacterium perishing phenomenon in the liquid by the cold plasma. In an experiment for investigating and revealing an operation mechanism of a surface sterilizing performance of the cold plasma in the solid culture medium, a surface sterilizing performance of the cold plasma was observed in a wider range as a length of the capillary tip becomes smaller. This means that activity decreased as the sterilization factors formed in the plasma generating part became more distant from the generation part. Meanwhile, when the tip is a micro-capillary tip, no sterilization performance was observed even when a length of the tip was decreased to 2 cm, and this hints that a factor that influenced the sterilization performance of the cold plasma was present in addition to a distance between the plasma generating part and the oral microorganisms that were to be perished.

In an experimental result for investigating and revealing a surface sterilizing mechanism of the cold plasma using two kinds of meshes in the experiment of the present disclosure, the solid culture medium surface sterilizing performance of the cold plasma did not greatly influence presence of the meshes of the dielectric material whereas the sterilization performance was considerably inhibited by the grounded electric mesh that may remove charge particles such as electrons and ions. This means that the oral microorganism surface sterilizing performance in the solid culture medium by the cold plasma was made not by H₂O₂ but by charged particles, unlike the sterilization performance in the liquid culture medium.

A lower concentration of H₂O₂ was observed in the culture medium for which the cold plasma was treated after the two kinds of meshes were installed than in the culture medium for which the cold plasma was treated while no mesh was installed. It was determined that the result was caused because a slightly small amount of OH radicals that were direct causes of formation of H₂O₂ of the culture medium were delivered to the culture medium while a flow of gases discharged from the cold plasma device became slower due to the mesh. However, because no change in the concentration of H₂O₂ was observed according to the kinds of the meshes, the result could mean that the difference between the oral microorganism perishing performances of the cold plasma according to the kinds of the meshes was not due to H₂O₂.

Furthermore, a possibility of a BRIC being inhibited was discussed because the sterilization performance time of charged particles of the cold plasma that was a main factor in the surface sterilizing performance of the cold plasma in the solid culture medium was very short. A strong sterilization ability was observed only at a narrow portion when the cold plasma was treated while being fixed to one point for 5 minutes, whereas an inhibition zone became wider when while a wider portion was treated while being moved for the same period of time. This means that the main factor that caused the oral bacterium surface sterilizing performance of the cold plasma flowed along the gas and then performed the sterilization operation only at a portion, with which the cold plasma directly collided, and the sterilization ability became weaker in a process of the cold plasma being diffused after the collision.

Finally, to pursue a measure for maintaining the surface sterilizing performance of the cold plasma in the solid culture medium even when the tip is mounted, the surface sterilizing performance of the solid culture medium by the cold plasma in a state, in which tips having various thicknesses, lengths, and shapes (straight or bent) was discussed. As a result, different S. mutans sterilizing performances by the cold plasma were observed according to the kinds of the tips mounted on the cold plasma device. In particular, it could be identified that the surface sterilizing performance of the cold plasma was increased as a gauge of the tip was lower, that is, as a pore size of the tip was larger, and as the length of the tip was smaller.

It was considered that the lowest surface sterilizing performance was observed when the capillary tip and the micro-capillary tip were mounted on the cold plasma device because the capillary tip had 27 gauges and the micro-capillary tip had 32 gauges whereby the pore sizes of the tips were too small and the lengths of the tips were large as well. The fact that the oral microorganism surface sterilizing performance of the cold plasma was became weaker due to the electric grounded mesh means that main charge particles of the cold plasma device could be argon ions or electrons.

It was considered that a phenomenon, in which the surface sterilizing performance was hindered according to the gauges of the tips mounted on the cold plasma device, could be caused because the argon ions that flowed together with the flows of the argon gas and the electrons caused a bottleneck phenomenon as the pore size of the tip became smaller, and accordingly, vortices were caused in the interior of the tip whereby two factors collided with each other and disappeared. It was considered that the surface sterilizing performance of the cold plasma became weaker because a possibility of bacterium perishing activities due to the argon ions and electrodes could become weaker when the length of the tip was two long even when the pore size of the tip was large. The fact that the phenomenon, in which H₂O₂ of the liquid culture medium due to the cold plasma was increased when the tips were mounted, did not coincide with the surface sterilizing performances for the tips also hinted that the surface sterilizing performance of the cold plasma was not due to OH radicals.

Consequently, according to the present disclosure, to dentally apply various medical performances of the plasma, a non-ozone cold plasma based dental disease treating device that is a new plasma generating device that maximally inhibits generation of ozone such that a respiratory system is safe while maintaining a low temperature, by which tissues of an oral cavity is not destructed was developed. The cold plasma generated according to the embodiment of the present disclosure effectively perished four main species of pathogenic bacteria that were present in the oral cavity.

It could be identified that the dental disease treating device according to the embodiment of the present disclosure immediately formed BRIC (clear zone) when the four species of bacteria cultivated in the solid culture medium were treated, and it was identified that an amount of generated ozone represented a very low value of 0.05 ppm and the device was a non-ozone cold plasma (NCP) device. Furthermore, it was identified that the dental disease treating device according to the embodiment of the present disclosure remarkably reduced the value of ozone and also reduced the size of the BRIC as plasma was concentrated in a local portion when the tip part was mounted.

It could be identified that all of the four species of pathogenic bacteria were decreased by 4 log or more when the plasma was treated on the oral microorganisms of the liquid culture medium by using the dental disease treating device according to the embodiment of the present disclosure, and it was identified that the sterilization performance for the liquid culture medium was maintained when the tip part was mounted. It was identified that the sterilization performance for the liquid culture medium of the dental disease treating device according to the embodiment of the present disclosure was due to an increase of the concentration of H₂O₂ of the culture medium.

Furthermore, the sterilization mechanism of the cold plasma was hydrogen peroxide, and the surface sterilization in the solid culture medium is a phenomenon due to charged particles generated when the cold plasma. Furthermore, it was identified that the sterilization ability of the cold plasma was maintained even when a tip of a specific condition was mounted on the cold plasma device. The sterilization performance for the solid culture medium by the dental disease treating device according to the embodiment of the present disclosure was not due to H₂O₂ but electrically charged elements, and it could be identified that the sterilization performance was inversely proportional to the length of the tip part mounted on the dental disease treating device and was proportional to the diameter of the passage of the tip part. As described above, the dental disease treating device according to the embodiment of the present disclosure showed a high perishing performance for various oral microorganisms, and could be utilized as a plasma generating device for an oral cavity due to a small amount of ozone.

An experiment for identifying a safety for the oral cells of a non-ozone cold plasma (NCP) generated by the dental disease treating plasma device according to the embodiment of the present disclosure was performed. To achieve this, it was identified whether the cells had toxics twenty four hours after the NCP was treated for one minute, three minutes, and five minutes on human gingival fibroblasts (HGF) that were oral periodontal cells, through a sulforhodamine B (SRB) assay.

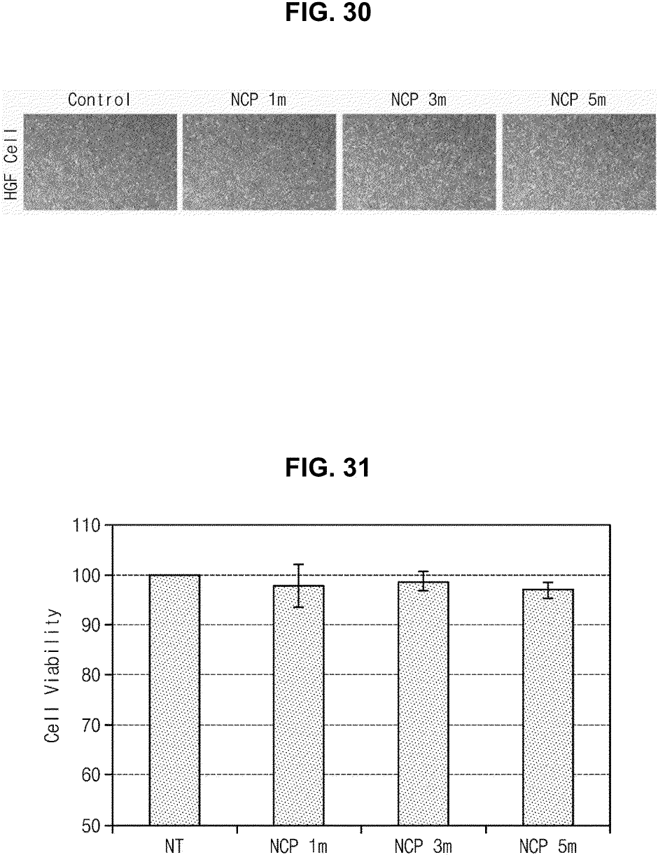

FIG. 30 is a picture of oral periodontal cells, on which the non-ozone cold plasma treatment was performed by the dental disease treating plasma device according to an embodiment of the present disclosure. FIG. 31 is a view illustrating a cell viability measurement result of oral periodontal cells, on which the non-ozone cold plasma treatment was performed by the dental disease treating plasma device according to an embodiment of the present disclosure.

It was identified from FIGS. 30 and 31 that the non-ozone cold plasma (NCP) generated by the dental disease treating plasma device according to the embodiment of the present disclosure did not induce perishing of cells even though it is treated for the oral cells for five minutes, and through this, a safety for the oral cells was verified.

An experiment for identifying an influence of the non-ozone cold plasma (NCP) generated by the dental disease treating plasma device according to the embodiment of the present disclosure on pseudo periodontitis inflammations that appeared in the oral cells was performed. An inflammation reaction that was similar to the periodontitis was caused by treating P. gingivalis originated lipopolysaccharide (PG-LPS) that was a causasive organism in HGF cells. The NCP was directly treated for the cells, in which the PG-LPS was treated, for one minute, three minutes, and five minutes, and then an additional cultivation was made for two hours, and then a RT-PCR and a Western blot experiment were performed by using the cell samples.

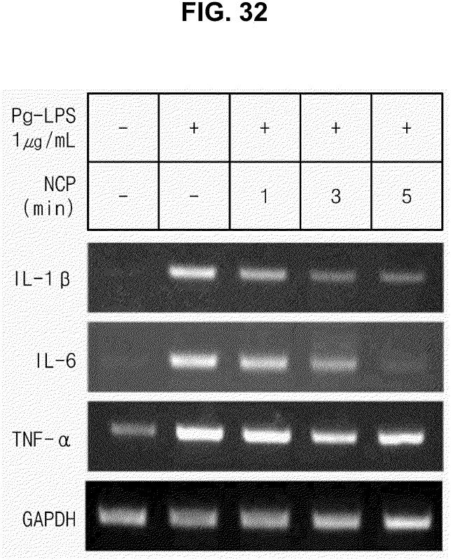

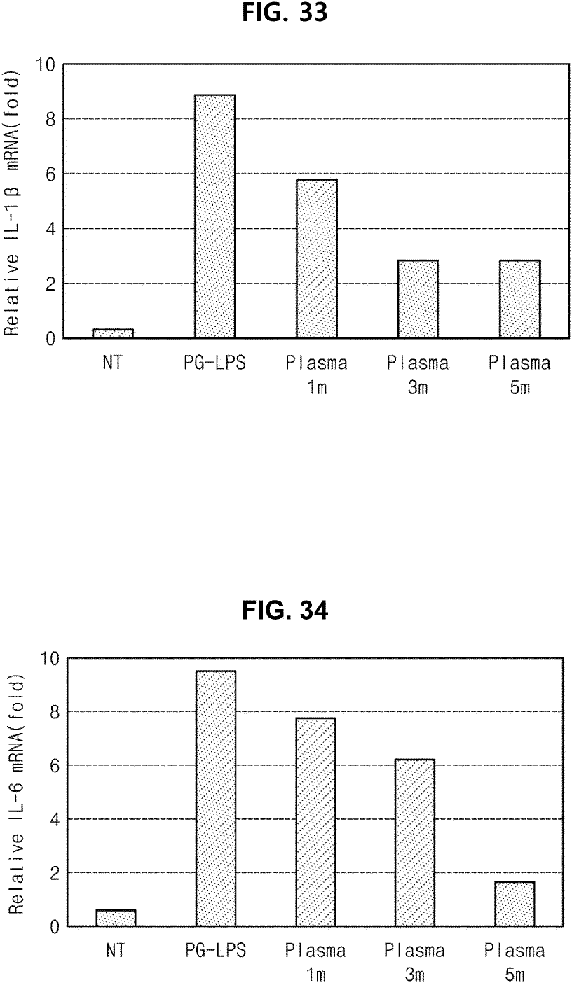

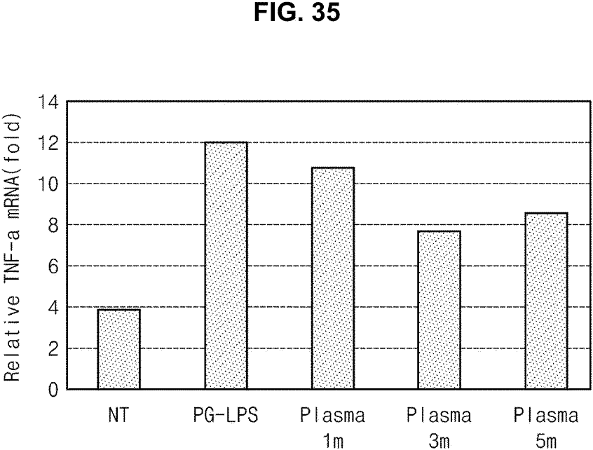

FIG. 32 is a view illustrating changes in an inflammation inducing factor during the non-ozone cold plasma treatment by the dental disease treating plasma device according to an embodiment of the present disclosure. FIG. 33 is a view illustrating changes in a 1L-1β mRNA gene expression inhibiting effect during the non-ozone cold plasma treatment by the dental disease treating plasma device according to an embodiment of the present disclosure. FIG. 34 is a view illustrating changes in a 1L-6 mRNA gene expression inhibiting effect during the non-ozone cold plasma treatment by the dental disease treating plasma device according to an embodiment of the present disclosure. FIG. 35 is a view illustrating changes in a TNF-amRNA gene expression inhibiting effect during the non-ozone cold plasma treatment by the dental disease treating plasma device according to an embodiment of the present disclosure.

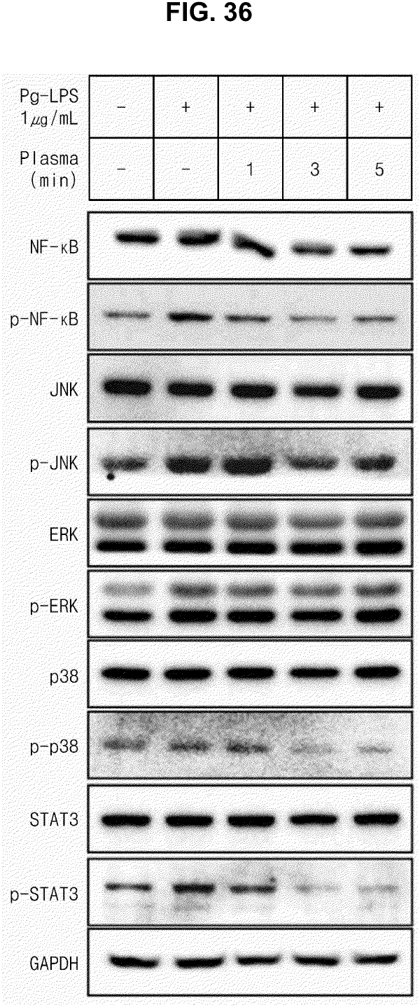

Referring to FIGS. 32 to 35, it could be identified that revelation of IL-1β, IL-6, and TNF-a genes that were inflammation causing factors, which were increased by the PG-LPS, was effectively inhibited by the NCP. FIG. 36 is a view illustrating changes in proteins during the plasma treatment by the dental disease treating plasma device according to an embodiment of the present disclosure. It is considered that the above-described inflammation causing factor revelation inhibiting phenomenon was caused because the functions of NF-kB and STAT-3 proteins that were increased by PG-LPS were effectively inhibited by the NCP. It is hinted that the above experimental result showed that the NCP could be applied to inhibition of other oral inflammations as well as inhibition of periodontitis inflammations because NF-kB and STAT-3 proteins were applied as important inflammation causing factors in inflammation diseases in various oral cavities.

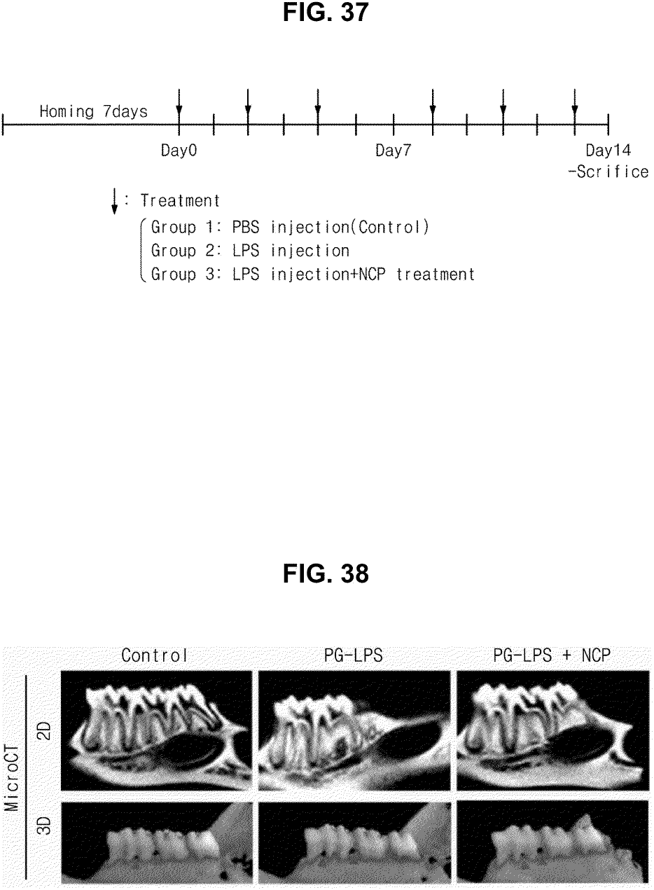

Next, an experiment for verifying an oral disease treatment performance (verifying a periodontitis treatment performance) of the dental disease treating device according to the embodiment of the present disclosure will be described. Verification using a periodontitis disease animal model was performed to identify whether the inflammation inhibiting performance in the oral cavity by the NCP could be used for an actual inflammation oral disease treatment. FIG. 37 is a view illustrating an experimental process for verifying a periodontitis treatment performance by the dental disease treating device according to an embodiment of the present disclosure.

As illustrated in FIG. 37, the experiment groups were divided into three groups of a normal tooth group (Group 1), a periodontitis group (Group 2), and a periodontitis + NCP group (Group 3), PG-LPS was inoculated into the first molar in the lower jaw three time per week for two weeks, a total of six times. The NCP was treated for the corresponding portion for five minutes in a state, in which the tip is mounted on the plasma device immediately after the inoculation of the PG-LPS.

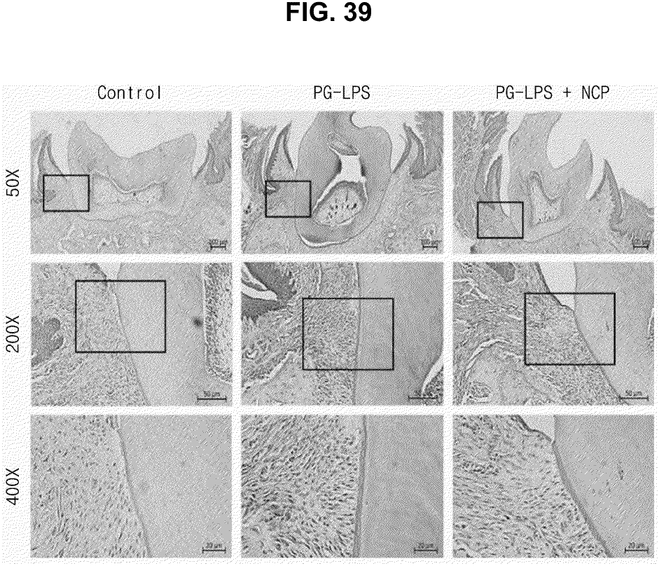

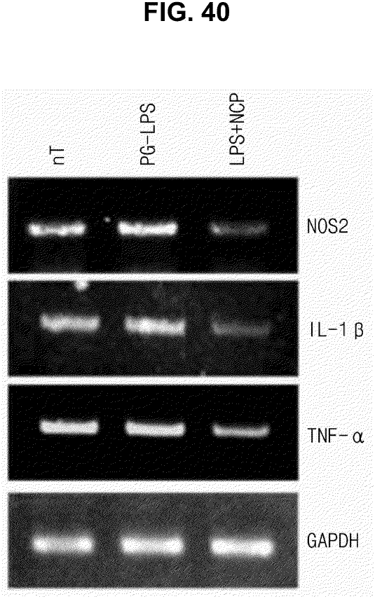

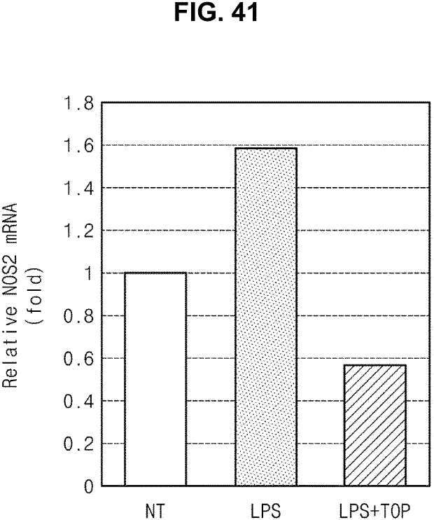

FIG. 38 is a micro CT analysis result using oral tissues extracted after an animal experiment was finished. FIG. 39 is a view illustrating a hematoxylin-eosin dyeing result using oral tissues after an animal experiment was finished. FIGS. 40 and 41 are views illustrating an RT-PCT study result using RNAs extracted from an experimental tissue.

In a micro CT analysis result using the tissues of the oral cavity extracted after the animal experiment was finished, as illustrated in FIG. 38, it could be identified that the NCP effectively inhibited an alveolar bone crushing phenomenon that appeared in the PG-LPS.

In a hematoxylin-eosin dyeing result using the extracted tissues of the oral cavity, as illustrated in FIG. 39, it could be identified that the density of the periodontal cells increased by the PG-LPS also was described by the NCP.

Furthermore, in an RT-PCT study using the RNA extracted from the experimental tissues, as illustrated in FIG. 40, it could be identified that revelation of IL-1β and TNF-a that are inflammation causing factors increased by the PG-LPS, and a NOS-2 gene that causes crushing of the teeth was effectively inhibited.

Finally, an immunohistochemistry (IHC) was performed on a CD68 protein that is a marker of a macrophage by using tissues of an animal experiment. FIG. 42 is a view illustrating an IHC result performed on the CD68 protein by using the tissues of an animal experiment. Referring to FIG. 42, in a result obtained by performing an IHC for a CD68 protein by using tissues of an animal experiment, a phenomenon, in which a density of macrophage cells were increased in a denticulate ligament, on which the PG-LGS was treated, was discovered, but it could be identified that a phenomenon, in which a density of macrophage cells was increased, could be effectively inhibited during the NCP treatment.

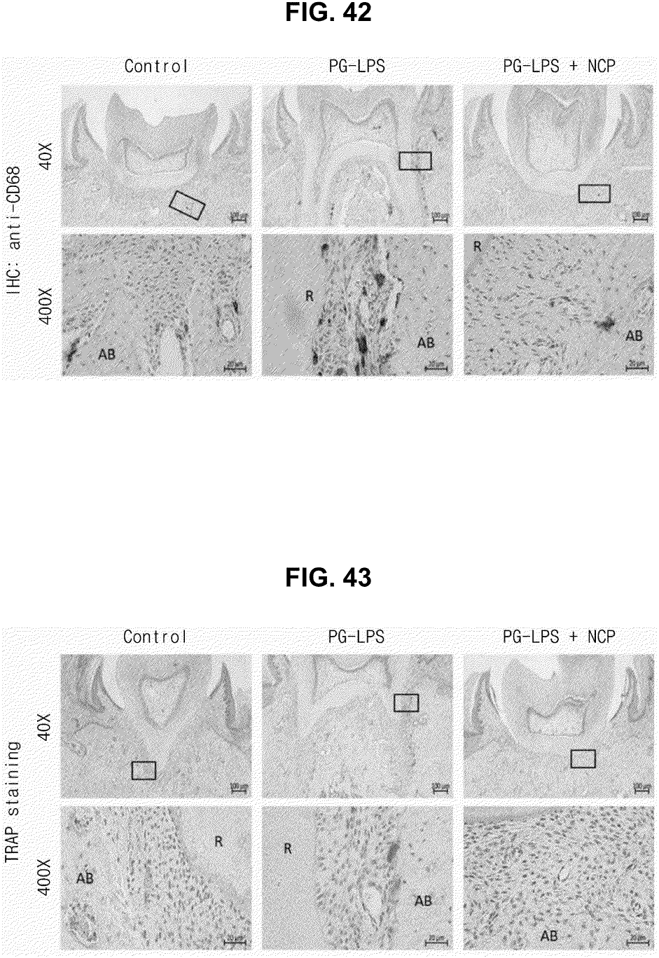

A TRAP assay that is a dyeing method for knowing activities of osteoclasts that are cells that destruct bones by using tissues of the animal experiment was carried out. FIG. 43 is a view illustrating a TRAP assay performance result using a tissue of an animal experiment. It also could be identified in FIG. 43 that osteoclast activities, in which the NCP was increased by the PG-LPS, were effectively inhibited. In the final summery, the results of the animal experiment hints that the NCP may effectively inhibits various inflammation reactions that appear in the oral tissues.

According to the embodiments of the present disclosure, high-density plasma that is required for a treatment of a dental disease, such as periodontitis, may be generated and generation of ozone as well may be minimized.

In addition, according to the embodiments of the present disclosure, various dental diseases may be effectively treated while a safety of oral cells is secured.

The advantageous effects of the present disclosure are not limited to the above-mentioned ones, and the other advantageous effects will be clearly understood by an ordinary person skilled in the art to which the present disclosure pertains.

The above detailed description exemplifies the present disclosure. Furthermore, the above-mentioned contents describe the exemplary embodiment of the present disclosure, and the present disclosure may be used in various other combinations, changes, and environments. That is, the present disclosure can be modified and corrected without departing from the scope of the present disclosure that is disclosed in the specification, the equivalent scope to the written disclosures, and/or the technical or knowledge range of those skilled in the art. The written embodiment describes the best state for implementing the technical spirit of the present disclosure, and various changes required in the detailed application fields and purposes of the present disclosure can be made. Accordingly, the detailed description of the present disclosure is not intended to restrict the present invention in the disclosed embodiment state. Furthermore, it should be construed that the attached claims include other embodiments.

What is claimed is:

1. A dental disease treating device that generates plasma for treating a dental disease, the dental disease treating device comprising:
a handpiece body having a space part in an interior thereof;
a plasma generating part mounted in the space part of the handpiece body, and configured to generate the plasma by exciting a gas in a plasma generating space in an interior thereof; and
a tip part detachably coupled to an end nozzle of the handpiece body or the plasma generating part,
wherein the tip part includes a passage for irradiating the plasma to a local portion while inhibiting oxygen from being introduced into the plasma generating space.

2. The dental disease treating device of claim 1, further comprising:
a grip part mounted to surround an outer peripheral surface of the handpiece body, and coupled to the handpiece body to be separable.

3. The dental disease treating device of claim 2, wherein a tip coupling part is formed at an end of the handpiece body, the tip coupling part is provided at an inner diameter portion of a tip end of the grip part, and the end nozzle is formed in an interior of the tip coupling part.

4. The dental disease treating device of claim 1, wherein the tip part has a Luer connector structure.

5. The dental disease treating device of claim 1, wherein the tip part includes:
a tip body having a cylindrical shape;
a plasma delivery pipe communicated with one side of the tip body, and having a cylindrical shape that is thinner than the tip body; and
a coupling hole formed on an opposite side of the tip body to be coupled to the end nozzle.

6. The dental disease treating device of claim 1, wherein the tip part inhibits generation of ozone when the plasma is generated, by preventing ambient air from penetrating into the plasma generating space.

7. The dental disease treating device of claim 1, wherein the plasma generating part includes:
a cylindrical body having an interior space;
an inner electrode inserted into the interior space of the body, and including a cylindrical electrode body; and
an outer electrode formed on an outer peripheral surface of the body through metalizing.

8. The dental disease treating device of claim 7, wherein a gas introduction pipe including a first passage, through which the gas for generating the plasma is introduced, is connected to a rear end of the electrode body, and a tip end of the electrode body has a conical shape.

9. The dental disease treating device of claim 8, wherein the first passage is communicated with an interior passage of the electrode body, and a plurality of gas discharge holes for discharging the gas introduced through the first passage toward the plasma generating space are radially formed in the electrode body.

10. The dental disease treating device of claim 7, wherein a first coupling part having a first opening is formed at one end of the body, a second coupling part having a second opening is formed at an opposite end of the body, a coupling recess that is to be coupled to the body is formed in the second coupling part, and a coupling boss formed on an inner surface of the handpiece body is coupled to the coupling recess.

11. The dental disease treating device of claim 7, wherein the inner electrode is grounded, and an AC voltage for exciting the gas is applied to the outer electrode.

12. The dental disease treating device of claim 7, wherein the plasma generating part further includes:
an elastic pad interposed between a second coupling part of the body, and a flange part of the inner electrode, and
wherein the elastic pad seals an aperture between the body and the inner electrode such that the gas is neither discharged nor introduced through the aperture between the body and the inner electrode.

13. The dental disease treating device of claim 7, wherein the body, the outer electrode, the inner electrode, and the tip part are designed to satisfy parameters of Equations 1 to 6 as follows such that a volume of the generated plasma and an acceleration distance of electrons are increased, high-density plasma is effectively delivered to an outer area, and generation of ozone is inhibited,
$$0.5 \leq \text{L1/L2} \leq \text{1}$$
$$0.5 \leq \text{D2/D1} \leq \text{0} \text{.8}$$
$$0.5 \leq \text{L3/L2} \leq \text{1}$$
$$0.5 \leq \text{L3/L2} \leq \text{1}$$
$$16 \text{gauge} \leq \text{D3} \leq \text{20 gauge}$$
$$2 \text{cm < L4 < 3} \text{.5 cm, and}$$
wherein in Equations 1 to 6, L1 is a length of the outer electrode, L2 is a length of the inner electrode, L3 is a distance between a front end of the inner electrode and a front surface of the body, L4 is a length of the tip part, D1 is a diameter of an inner passage of the body, D2 is a diameter of the inner electrode, and D3 is a minimum diameter of a passage of the tip part.

14. The dental disease treating device of claim 13, wherein the length of the inner electrode is 10 mm to 20 mm, the length of the outer electrode is 5 mm to 20 mm, the diameter of the inner electrode is 2 mm to 4 mm, the diameter of the inner passage of the body is 3 mm to 5 mm, and a distance between a front end of the inner electrode and a front surface of the body is 1 mm to 4 mm.

15. A method for treating a dental disease, by which the dental disease of a target object is treated by using the dental disease treating device of claim 1.
